(19) United States
(12) Patent Application Publication
(10) Pub. No.: US 2009/0192518 A1
(43) Pub. Date: Jul. 30, 2009

(54) APPARATUS AND METHOD FOR LOADING AND DELIVERING A STENT HAVING IMPROVED HANDLES TO CONTROL RELATIVE CATHETER COMPONENT MOVEMENT

(75) Inventors: John Golden, Norton, MA (US); Mark Wood, Shrewsbury, MA (US); John McWeeney, Brighton, MA (US); Gary Leanna, Holden, MA (US)

Correspondence Address: HOFFMANN & BARON, LLP, 6900 JERICHO TURNPIKE, SYOSSET, NY, 11791, US

(73) Assignee: BOSTON SCIENTIFIC SCIMED, INC., Maple Grove, MN (US)

(21) Appl. No.: 12/358,295

(22) Filed: Jan. 23, 2009

Related U.S. Application Data
(60) Provisional application No. 61/023,233, filed on Jan. 24, 2008.

(51) Int. Cl. A61F 2/84; A61B 17/00

(52) U.S. Cl. 606/108

(57) ABSTRACT

A catheter handle assembly for delivering stent includes a proximal handle attached to an axially elongated inner member with a distal tip and an intermediate handle attached to an axially elongated intermediate tube with a stent basket at its end. The intermediate tube overlies at least a portion of the inner member. A distal handle is attached to an axially elongated external member overlying at least a portion of the intermediate tube are disclosed.

### CROSS-REFERENCE TO RELATED APPLICATIONS

This Application claims the benefit of U.S. Provisional Application No. 61/023,233, filed Jan. 24, 2008, the contents of which are incorporated herein by reference.

### FIELD OF THE INVENTION

This invention relates to a delivery assembly and method for transporting, loading and delivering a stent in a bodily passageway. More particularly, this invention relates to systems and methods for loading and delivering radially distensible stents, including polymeric and non-polymeric stents.

### BACKGROUND OF THE INVENTION

An intraluminary prosthesis is a medical device used in the repair and/or treatment of diseases in various body vessels is a stent. A stent is generally a longitudinal tubular device formed of biocompatible material useful to open and support various lumens in the body. For example, stents may be used in the bodily vessel, such as in the coronary or peripheral vasculature, esophagus, trachea, bronchi colon, biliary tract, urinary tract, prostate, brain, as well as in a variety of other applications in the body. These devices are implanted within the vessel to open and/or reinforce collapsing or partially occluded sections of the lumen.

Stents generally include an open flexible configuration such as helically wound coils with undulations or zig-zags therein, slotted stents, ring stents, braided stents and open mesh wire stents, to name a few. Super-elastic materials and metallic shape memory materials have also been used to form stents. This flexible configuration allows the stent to be inserted in a radially compressed state through curved vessels. Once properly positioned adjacent to the damaged vessel, the stent is radially expanded so as to support and reinforce the vessel. Radial expansion of the stent may be accomplished by inflation of a balloon attached to the catheter or the stent may be of the self-expanding variety which will radially expand once deployed. Once the stent is implanted, the delivery catheter is withdrawn from the patient.

With any of these systems, a sheath may be provided over the distal end of the catheter to protect the components on the distal end, such as a balloon, a stent, an array of electrodes, and the like. In some embodiments, the sheath may be advanced distally over the proximal end of the catheter until it covers the distal end and its components, or, alternatively, the distal end of the catheter may be introduced into the sheath, and advanced until it is proximate the distal end of the sheath. Once the distal end of the catheter is properly positioned at a desired location within a body lumen, the sheath may be retracted to expose the distal end of the catheter. After treatment, the sheath may be advanced back over the distal end of the catheter or the catheter may be withdrawn back into the sheath, and the entire device withdrawn from the patient.

To cause the sheath to retract, the proximal end of the sheath outside the patient may simply be pulled while holding the catheter in a fixed position. This, however, may not provide very precise control of the retraction of the sheath. Moreover, in such devices, it is possible to advance the sheath in the distal direction during and after deployment of the device, such as a stent, on the distal end of the catheter. This distal movement may result in the improper placement and unwanted movement of the deployed device. This distal movement of the sheath is particularly problematic in the deployment of stents or other tubular prostheses. Accordingly, there is a need for more intuitive, simpler, and/or less expensive devices for controlling catheter-sheath systems.

Although stent delivery systems are well-known in the art, the assembly of such delivery systems is often complicated. Unlike most metallic self-expanding stents, the plastic stents have a tendency to permanently deform or lose some of their ability to self-expand when stored in a compressed state for a prolonged period of time. These stents are therefore preferably loaded into the stent delivery system shortly before being implanted in a patient.

However, such loading step just prior to the actual surgery often involves numerous steps and requires the use of multiple components (e.g., tools and fixtures) that are not part of the stent delivery system. Also, even with these added devices, the practitioner is often required to finish the loading process by pushing the stent into the delivery system by hand. Loading a stent in this way is therefore often difficult, time-consuming and has the potential to damage the stent. Accordingly, there is a need for simplified methods of on-site loading of a stent into stent delivery systems, while minimizing the risk of damaging the stent in the process.

### SUMMARY OF THE INVENTION

The present invention is directed to a method and system for delivering a self-expanding stent into a body lumen. In particular, the present invention relates to an assembly and a method for loading and delivering a stent in combination with a stent delivery catheter device, as well as to overall stent delivery systems.

In one aspect of the present invention, a stent loading and deployment device may be provided. The device may include an axially elongated external member having opposed proximal and distal ends; and an axially elongated inner member having opposed proximal and distal ends and slidably disposed within the axially elongated external member. When the distal ends of the axially elongated external member and the axially elongated inner member are axially aligned, a stent deployment region can be defined there in between. An intermediate member having opposed proximal and distal ends can be slidably disposed between the external member and the inner member. In some embodiments, the distal end of the intermediate member may be slidable to a distal position past the distal end of the external member for receiving a stent and may be further slidable toward the proximal end of the external member to a location past the stent deployment region for disengagement of the stent from the intermediate member. The external member, the inner member and/or the intermediate member may be axially movable or slidable independently of each other. They may also be axially movable or slidable in concert in either total or in different combinations of pairs. For example, the distal end of the intermediate member may be slidable to a distal position past the distal end of the external member while the positions of the inner member and the external member are kept constant or relatively constant. The intermediate member may be further slidable toward the proximal end of the external member to a location past the stent deployment region while the positions of the inner member and the external member are kept constant or relatively constant.

The device may further include a stent basket having opposed proximal and distal ends. In some embodiments, the proximal end may be securely disposed to the distal end of the intermediate member. The stent basket may have a truncated-conical shape, outwardly diverging in a distal direction from its proximal end. The stent basket may be a thin film which can collapse such that the stent basket may be slidably contained within the external member, or may be a radially distensible member which can collapse such that the stent basket may be slidably contained within the external member. In some embodiments, the stent basket may be composed of a polymeric material. The stent basket may include, in part or substantially, braided polymeric filaments. The braided filaments may be contained within a thin polymeric film. The intermediate member may be an elongate tubular device. The stent basket may comprise metals, polymers, or combinations of both.

The device may further include a tubular band disposed toward the distal end of the inner member for releasably securing a stent in the stent deployment region between the inner member and the external member. In some embodiments, the external member can be slidable toward a proximal position for releasing the stent from the stent deployment region. Typically, the external member may slide while the inner member and the stent basket may be fixed or not in substantial movement.

The device may further include a distal handle disposed at the proximal end of the external member; a proximal handle may be disposed at the proximal end of the inner member; and an intermediate handle may be disposed at the distal end of the intermediate member. The intermediate handle may be axially disposed between the distal handle and the proximal handle. The distal handle may be axially disposed distal to the proximal end of the inner member. The handles may separated, mechanically mated, including temporarily mated or locked, and/or integrated to allow independent or non-independent axial movement or sliding of the external member, the inner member and the intermediate member.

The device of this aspect can be useful containing and releasing a radially distensible stent. The radially distensible stent may be a polymeric stent, including a braided stent. A graft, such as a covering, a liner, a film, a coating and combinations thereof, may be disposed over at least a portion of the stent. In some embodiments, the stent can be a braided polymeric stent and the graft may be a silicone coating or film. Further, the stent may be a multi-component stent (graft separate from stent) and may comprise metal.

In another aspect of the present invention, a stent loading and deployment system can be provided. The system includes a radially distensible stent; an external member having opposed proximal and distal ends; an inner member having opposed proximal and distal ends and slidably disposed within the external member, wherein, when the distal ends of the external member and the inner member can be axially aligned, a stent deployment region being defined there in between; and an intermediate member having opposed proximal and distal ends and slidably disposed between the external member and the inner member; wherein the distal end of the intermediate member may be slidable to a distal position past the distal end of the external member for receiving the stent and can be further slidable toward the proximal end of the external member to a location past the stent deployment region for disengagement of the stent from the intermediate member.

A method for loading a stent into a delivery and deployment may also be provided with under the present invention, such method includes (i) providing a radially distensible stent having opposed proximal and distal ends; providing a delivery deployment device, the device including an external member having opposed proximal and distal ends; an inner member having opposed proximal and distal ends and slidably disposed within the external member, wherein, when the distal ends of the external member and the inner member may be axially aligned, a stent deployment region may be defined there in between; a stent basket having opposed proximal and distal ends, wherein the proximal end of the stent basket can be securely disposed to the distal end of the intermediate member; (ii) axially moving or sliding the distal end of the intermediate member to a distal position past the distal end of the external member; (iii) engaging the proximal end of the stent with the stent basket; (iv) axially moving or sliding the stent and the intermediate member toward the proximal end of the external member to radially compress the stent within the stent deployment region; and (v) axially moving or sliding the stent basket to a location past the stent deployment region for disengagement of the stent from the intermediate member.

The method may further include providing a tubular band disposed toward the distal end of the inner member for releasably securing the stent in the stent deployment region between the inner and external members. Moreover, the method may further include axially moving or sliding the external member toward a proximal position for releasing the stent from the stent deployment region. The method may yet further include providing a distal handle disposed at the proximal end of the external member; providing a proximal handle disposed at the proximal end of the inner member; and providing an intermediate handle disposed at the proximal end of the intermediate member, wherein independent axial movement of the external member, the inner member or the intermediate member can be achieved by manual manipulation of the handles.

These and other objectives, features, and advantages of this invention will become apparent from the following detailed description of illustrative embodiments thereof, which is to be read in connection with the accompanying drawings.

### BRIEF DESCRIPTION OF THE DRAWINGS

FIG. 1 is a cross sectional view of an embodiment of the present invention.

FIG. 2 is a cross-sectional view of distal part of the embodiment as shown in FIG. 1.

FIG. 3 is a top plan view of a stent of the present invention.

FIG. 4 is a cross sectional view of another embodiment of the stent of the present invention.

FIG. 5 is a cross sectional view of yet another embodiment of the stent of the present invention.

FIG. 6 is a cross sectional view of further yet another embodiment of the stent of the present invention.

FIG. 7 is a side plan view of further yet another embodiment of the stent of the present invention.

FIG. 8 is a side plan view of further yet another embodiment of the stent of the present invention.

FIG. 9 is a cross-sectional view of the embodiment shown in FIG. 1 with stent loaded.

FIG. 10 is a cross sectional view of the embodiment shown in FIG. 9 with the intermediate handle pulled toward the proximal handle.

FIG. 11 is a cross sectional view of the embodiment shown in FIG. 9 with the stent partially deployed.

FIG. 12 is a cross sectional view of the three handles of the embodiment shown in FIG. 1.

FIG. 13 is a cross sectional view of the handle as shown in FIG. 12 with intermediate handle distally located.

FIG. 14 is a side plan view of the embodiment shown in FIG. 1 prior to loading of a stent.

FIG. 15 is a side plan view of the embodiment shown in FIG. 14 with a stent partially held by a basket.

FIG. 16 is a side plan view of the basket and the stent of the embodiment of FIG. 1.

FIG. 17 is a side plan view of the basket and the stent of the embodiment of FIG. 1.

FIG. 18 is an exploded plan view of each tubular member separate from each other.

FIG. 19 is a side plan view of yet another embodiment of the basket as shown in FIG. 18.

FIG. 20 is a side plan view of further yet another embodiment of the basket.

FIG. 21 is a side perspective view of axially elongated inner member of the present invention.

FIG. 22 is a side perspective view of the proximal handle of the present invention.

FIG. 23 is a side plan view of the proximal handle as shown in FIG. 22.

FIG. 24 is a cross sectional view of the bevel edge of the tubular member of the present invention.

FIG. 25 is a cross sectional view of the handles of the present invention.

FIG. 26 is a side perspective view of the basket retractor handle of the present invention.

FIG. 27 is a side perspective view of the intermediate handle release mechanism of the present invention.

FIG. 28 is a side perspective view of yet another embodiment of the intermediate handle with winged feature.

FIG. 29 is a side cutaway view of the proximal handle with mechanisms molded therein.

FIG. 30 is a side plan view of the present invention with the intermediate handle at an initial position.

FIG. 31 is a side plan view of the embodiment shown in FIG. 1 with the distal handle at a position away from the intermediate handle.

FIG. 32 is a side perspective view of the embodiment shown in FIG. 1 with the intermediate handle fully inserted into the proximal handle.

FIG. 33 is a side perspective view of the embodiment shown in FIG. 1 with the distal handle at a position close to the proximal handle.

FIG. 34 is a side cross sectional view of the three handles of the embodiment of FIG. 1.

FIG. 35 is a side cross sectional view of yet another embodiment of the mechanism and handle interlock mechanisms.

FIG. 36 is a side perspective view of the embodiment with distal handle close to the intermediate handle.

FIG. 37 is a perspective view of the embodiment shown in FIG. 1 with intermediate handle having a button.

### DETAILED DESCRIPTION OF PREFERRED EMBODIMENTS

References herein to the term “distal” and variants thereof refer to a direction away from a practitioner of the subject invention, while references to the term “proximal” and variants thereof refer to a direction towards the operator of the subject invention. Accordingly, when the terms “distal” and “proximal” are used herein in the context of an assembly device or system that is being deployed within a body, such as a human body, by an operator, the term “distal” refers to a location within or near the body that is further within the body than a location that is “proximal” to the operator.

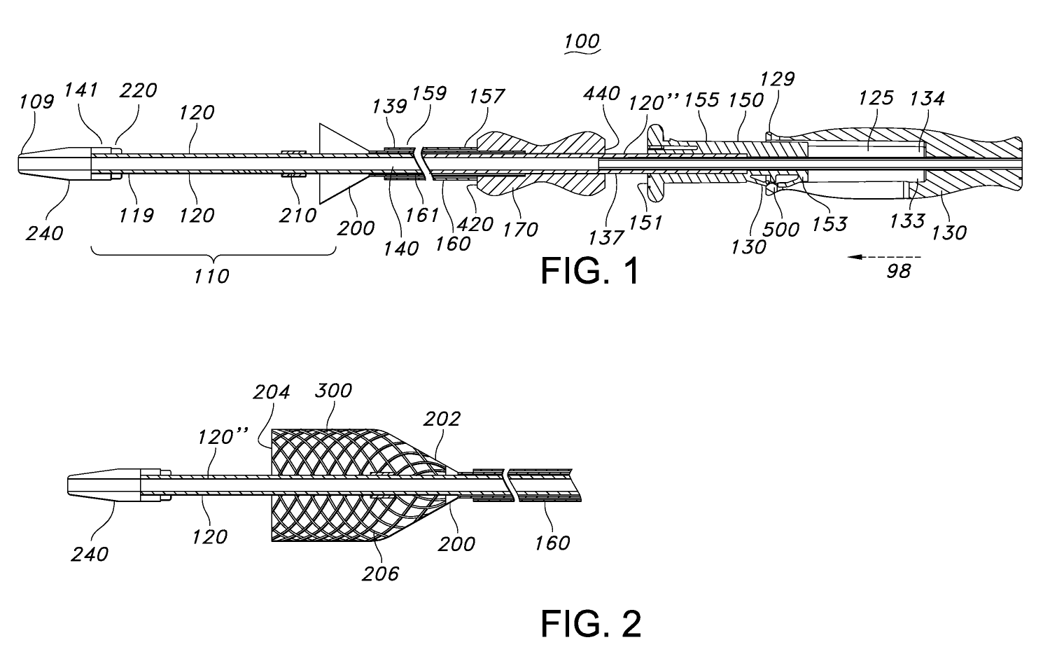

FIG. 1 is a cross-sectional view of a stent loading and delivery system 100 according to the present invention. The system 100, as depicted, may be particularly well suited for loading, transluminal delivery and intraluminal deployment of a radially self-expanding prosthesis, such as a stent and/or a stent-graft. The system 100 may include a catheter-type device with three elongated cylindrical members concentric about an axis and having opposed proximal and distal ends. The three members can be structured as follows: A flexible axially elongated inner member 120 (which may be constructed as a tube or a solid), that may include a support platform or stent holder 210 off which a stent can be delivered; an axially elongated tubular intermediate member 140 slidably containing the inner member 120 therewithin; and an axially elongated tubular external member 160 slidably containing the intermediate member 140 therewithin, wherein all members interrelate with each other as shown in FIG. 1. The members 120, 140, and 160 shall be described in detail below.

The members 120, 140, and 160 may be manipulated to cause changes to a stent deployment region 110 located at the distal end 109 of the delivery system 100. In particular, the stent deployment region 110 may be defined as a region between the distal end 159 of the external member 160 and distal end 119 of the inner member 120 when these axial ends 159, 119 are at a particular position farthest away from each other. In some embodiments, the stent holder 210 of the intermediate member 140 can slide to a distal position 141 past the distal end 159 of the external member 160 for receiving a stent 300. In addition, the stent holder 210 can slide in an opposite direction toward the proximal end 157 of the external member 160 to a location 161 past the stent deployment region 110 for disengagement of the stent 300 (which is depicted in FIG. 2) from the intermediate member 140.

The mechanical relationship between the members 120, 140, and 160 shall now be described. Each member of the system 100 may be defined and controlled at the proximal end by a respective handle as follows: A proximal handle 130 may be fixedly disposed at the proximal end 117 of the inner member 120; an intermediate (for basket retraction in some embodiments) handle 150 may be disposed at the proximal end 137 of the intermediate member 140; and a distal (for stent-deployment in some embodiments) handle 170 may be disposed at the proximal end 157 of the external member 160. Longitudinally, the distal handle 170 may in some embodiments be disposed furthest away from the practitioner in relation to other handles or away from the proximal end 117 of the inner member 120. The intermediate handle 150 may be disposed between the distal handle 170 and the proximal handle 130, which may be disposed closest to the practitioner.

The handles 130, 150, and 170 are displaceable longitudinally along the axis 98 relative to each other thereby enabling selective deployment and retraction of the stent 300 (shown in FIG. 15). In essence, manipulation or axial movement of the handles 130, 150 and 170 permits independent axial movement of the tubular members 120, 140, and 160, respectively. For example, the intermediate handle 150 may slide between distal and proximal positions 151, 153 so as to axially move the intermediate member 140. Such movement may be done while keeping the other handles 130, 170 fixed or relatively fixed to allow independent or substantially independent movement of the intermediate member 140. While the intermediate member 140 is moved, the inner member 120 and the external member 160 may remain fixed or relatively fixed.

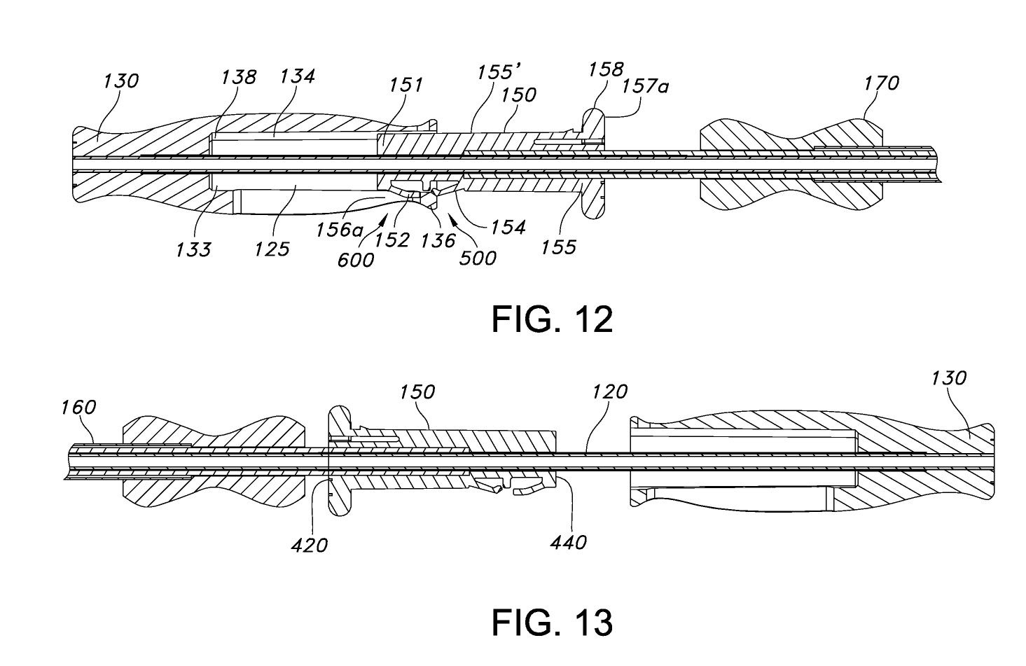

The present invention provides for the proximal handle 130 to cooperate with the intermediate handle 150. Proximal handle 130 can be designed to fully retract or receive a substantial portion of the intermediate handle 150. In some embodiments, as depicted in FIG. 12, the proximal handle 130 can be fabricated with an opening 132 and a receptacle 134 therein to receive a particular shaft portion 155′ of the intermediate handle 150. Although FIG. 12 may depict the opening 132 with a circular or oval shape and the receptacle 134 with a barrel shape, these shapes are presented for illustrative purposes only. The present invention encompasses other shapes besides the illustrated shapes for these members. The shaft portion of the intermediate handle 150 may be provided with a slidable shape (tubular or otherwise) to easily facilitate sliding into the receptacle 134. Additionally, a linear surface of the receptacle 134 may include a guide 138 and the intermediate handle 150 may include a corresponding track 156 to assist or guide the intermediate handle 150 to easily slide in and out and to prevent unwanted twisting. Alternatively, the end to end inner dimension of the receptacle 134 and the distance of the shaft 155 of the intermediate handle 150 may be fashioned with predetermined lengths. Thus shaped, the interior end 133 of the receptacle 134 may obstruct the proximal end of the intermediate handle 150 preventing the distal end of the intermediate handle 150 from engaging or colliding into the distal end of the proximal handle 130.

Additionally, as depicted in FIG. 12, the intermediate handle 150 in combination with the proximal handle 130 may include a locking mechanism 500 (shaped, for instance, with tactile detent). The locking mechanism 500 may be configured to control the distal positioning of the intermediate handle 150 with respect to the proximal handle 130 as shall be described in detail below. Furthermore, the intermediate handle 150 in combination with the proximal handle 130 may also include a handle interlock mechanism 600 that interlocks and prohibits a complete removal of the intermediate handle 150 from the receptacle 134. The handle interlock mechanism 600 may be shaped with tactile features, allowing the practitioner to control and lock the elements based on the practitioner's sensory perception.

The details of the mechanism 500 are described herein below. As depicted in FIG. 12, a section 131 of the opening 132 may include an overhang protrusion 136 located at a distal end position 129 of the proximal handle 130. In some embodiments, the protrusion 136 may be provided on the opposite side of the guide 138. However, it may also be placed anywhere alongside in the interior receptacle in congruence with the portions of the intermediate handle 150. The corresponding portions of the intermediate handle 150 may include a mechanism 154 (flexible in some embodiments) and a handle interlock 152 located at a position near the proximal end 151 of the intermediate handle 150. The flexible mechanism 154 can be shaped with a curved sloping extension that extends toward the proximal end of the intermediate handle and bends radially inwardly when an external force is applied. When the intermediate handle 150 is retracted into the receptacle 134, the mechanism 154 can be overcome by the protrusion 136.

Similarly, the handle interlock mechanism 600 can be formed. In particular, the handle interlock 152 can be formed with a protrusion that extends from a location adjacent to the proximate end of the intermediate handle 150. The shape of the handle interlock 152 can be designed with a surface region so that it will engage a region of the protrusion 136, thereby limiting further linear movement of the intermediate handle 150. Thus, the handle interlock 152 “captures” the intermediate handle 150. Such mechanism 600 can also prevent the intermediate handle 150 from moving the intermediate member 140 too far distally and prevent interference of the stent loading basket 200 with the stent holder 210. In some embodiments, the handle interlock 152, as well as the mechanism 154, may be provided on the proximal underside of the intermediate handle 150. However, similar to the placement of the protrusion 136, these mechanisms may be placed anywhere alongside the shaft 155 of the intermediate handle 150 in congruence with the location of protrusion 136.

The locking mechanism 500 and the handle interlock mechanism 600 can be enhanced by another mechanism for a stable state. Also seen in FIG. 12, a ledge 156a may be provided between the mechanism 154 and the handle interlock 152 so as to provide a recessed stable region for the protrusion 136. Thus formed, as the intermediate handle 150 protracts from a fully retracted position, the protrusion 136 may slide over the mechanism 154 and come to rest on the ledge 156a. Once the protrusion 136 rests on the ledge 156a, the handle interlock 152 catches and prevents the protrusion 136 from sliding further toward the proximal end 151 of the intermediate handle 150 and prevents the intermediate handle 150 from being dislodged completely. The position where the protrusion 136 rests on the ledge 156 defines the position where the two handles 130 and 150 become locked together and the positions of the members 120 and 140 are stationary relative to each other. This position also defines the initial stent loading position where the practitioner can easily place a stent for loading. This position allows the practitioner to load the stent, prior to completely retracting the intermediate handle 150 into the proximal handle 130. Therefore, the protrusion 136 in combination with the handle interlock 152 and the mechanism 154 provide a systematic control that not only stabilizes the position of the intermediate member 140, but also allows for proper stent placement. Thus shaped, the relative position and movement of the proximal and intermediate handles 130, 150 can be mechanically limited by the enclosed and bounded design of the handles 130, 150 and 170.

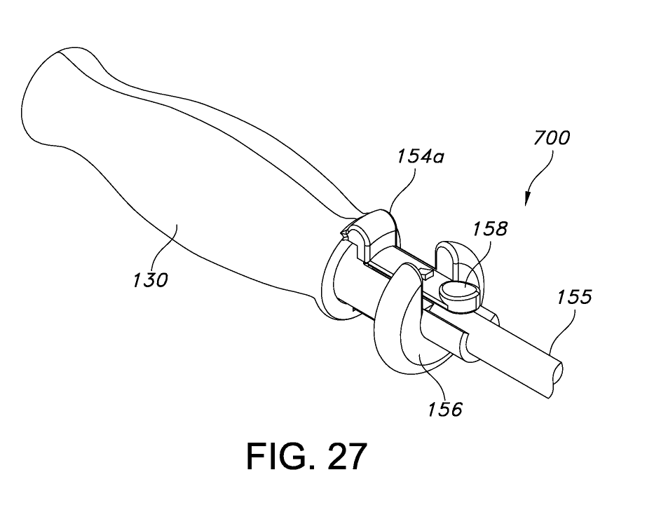

The protrusion 136, the handle interlock 152 and the mechanism 154 combination may be further enhanced in several different ways. For instance, the intermediate handle 150 may include a distal flange portion 157a that may serve as an alternative stop when the intermediate handle 150 is inserted into the receptacle 134. Furthermore, the flange portion 157a may be designed to slide neatly into a distal recessed region 125 on the proximal handle 130. Thus, the intermediate handle 150 can be flush within the proximal handle 130 when retracted, or can be designed to be less accessible as shown in FIG. 27. When the release mechanism 700 for the intermediate handle 150 is less accessible to the practitioner, it is less likely that the stent may be prematurely deployed.

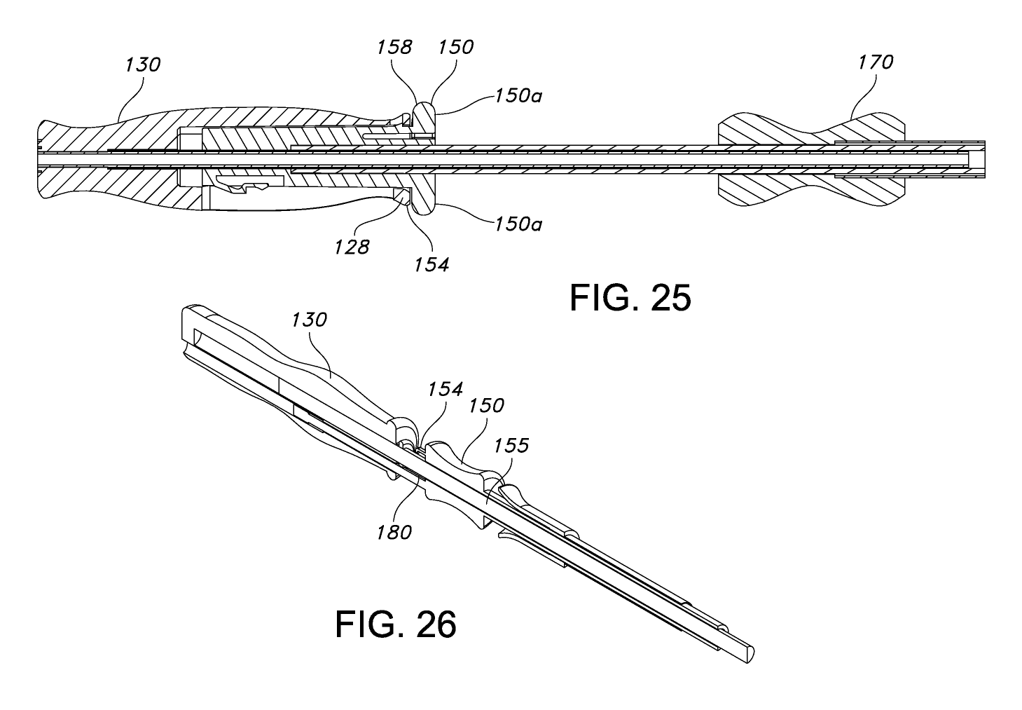

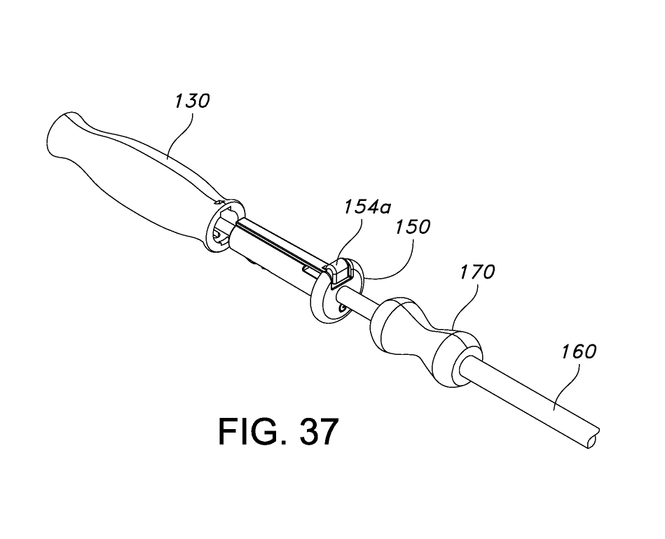

In addition, the flange portion 156a of the intermediate handle 150 may also include a release mechanism such as a button 158 that protracts and retracts the intermediate handle 150 in relation to the proximal handle 130. In particular, the release mechanism or button 158 actuates the depression of mechanism 154a (which may function as a detent) and disengages the intermediate handle 150 from the protrusion 136, thereby facilitating a full retraction into the proximal handle 130 when the stent needs to be loaded. FIG. 37 is a partial perspective view of the delivery system 100 showing the handles 130, 150, 170 and mechanism 154a, inter-related as shown. FIG. 26 depicts the details of the opening 132 and a substantial shaft portion 155 of the intermediate handle 150 immediately prior to being retracted into the receptacle 134. As depicted in FIG. 25, the intermediate handle 150 may also include the release mechanism 158 on the top portion 150a to actuate the movement of the mechanism 154 on the bottom portion 150b of the intermediate handle 150, thereby allowing the intermediate handle to fully retract into the proximal handle 130. In addition, the proximal handle 130 may include a flange portion 128 to cooperate with the release mechanism 158. Thus, the release mechanism 158 of the intermediate handle 150 can be designed to be flush with the flange portion 128 of the proximal handle 130 when retracted within the receptacle 134, or can be designed to be less accessible as shown in FIG. 27.

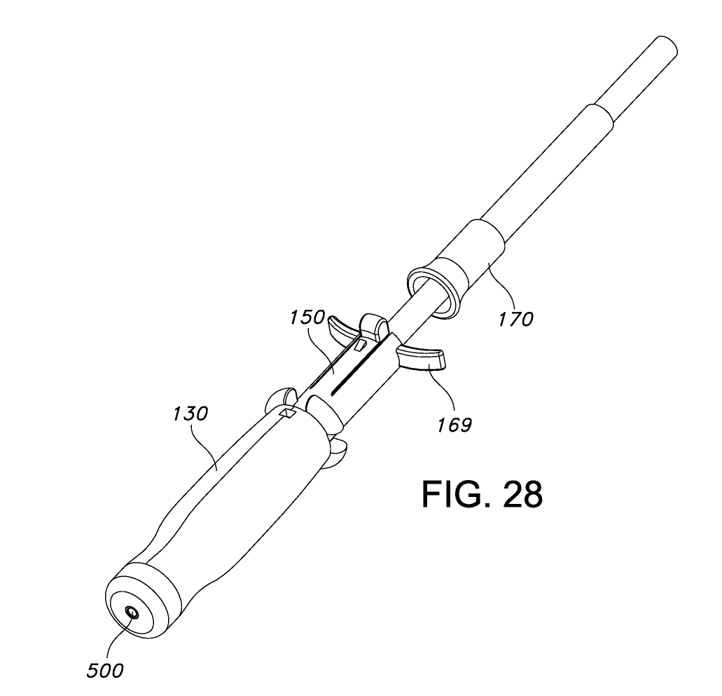

Several other features are envisioned by this invention. For instance, in an alternate form embodiment, the handle interlock 152 and mechanism 154 can be a single molded feature in the design of the intermediate handle 150, as depicted in FIG. 12. In some embodiments, as illustrated in FIG. 28, the intermediate handle 150 can have alternate features. For example, the intermediate handle 150 may include winged projections 169, in some embodiments at its distal end 153 to improve grip. The winged projections 169 may allow a practitioner to more easily grasp one of the projections 169 by utilizing his/her fingers or any other means. The intermediate handle 150 can have other alternate features to improve grip as well. For instance, in yet another embodiment of the delivery system 100 according to the current invention includes the position of the intermediate handle 150 being controlled by another controlling means such as a rod (not shown), that interlocks with the proximal handle 130. In some embodiments, the position of the intermediate handle 150 can be controlled by another grip such as a rod that interlocks with the proximal handle 130. Also, the retention of the intermediate handle 150 can be made permanent by features such that the handles cannot be separated if this feature is desirable. For instance, the handles may interlock with screw threads, ratchets, adhesives, friction, or other mechanisms as will be understood by a person of ordinary skill in the art.

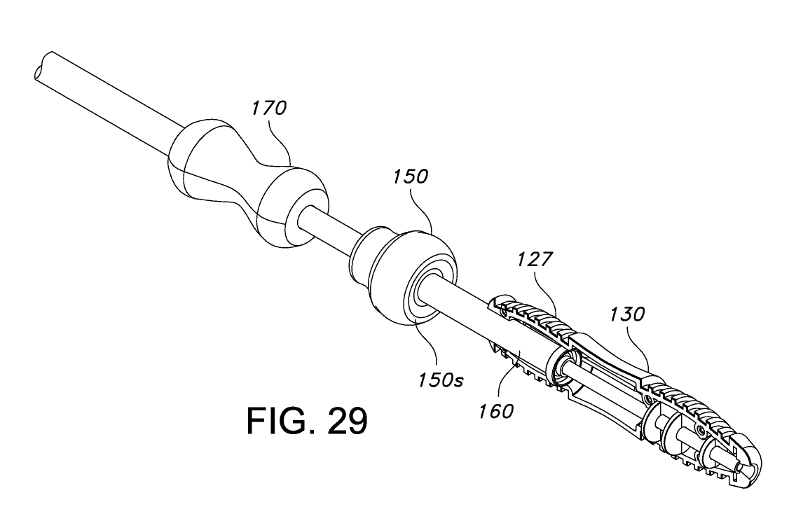

Additionally, each grip portion of the handles 130, 150, and 170 may also include additional inventive features. For instance, by molding ribs 127 onto the proximal handle 130 as depicted in FIG. 29, the proximal handle 130 can be designed to engage the intermediate handle 150 during the assembly process to improve attachment of the catheter tubular members. As depicted in FIG. 29, in some embodiments, the intermediate handle 150 may include a flange style or a knob-style stop 150s with a mechanical feature which correspondingly interacts with the proximal handle 130. Furthermore, the mechanism 154 for intermediate handle 150 can also be molded into the handle interior of the proximal handle 130 as depicted in FIG. 29. Thus shaped, the catheter's tube may pass through and can be secured in the ribs 127 of the proximal handle 130. In some embodiments of this invention, the proximal handle 130 may be designed to better grip and hold the intermediate handle 150 during the assembly process. Also, all handles may have softer polymers for grip or ergonomic designs for surface designs. For instance, molding ribs 127 may be fashioned onto the tubular members of the proximal handle 130 to improve securement into the intermediate handle 150. In addition, all mechanism may be molded in place. For instance, in some embodiments, mechanisms 154 for the intermediate handle 150 may also be molded into the interior of the handle 150.

As depicted in FIG. 1, the system 100 may further include a distal tip 240 disposed at the distal end of the inner member 120. Thus, the inner member 120 may be in some embodiments defined at one end by the distal tip 240 and at the other end by the proximal handle 130. Distal tip 240 may be symmetrically optimized for atraumatic insertion and withdrawal of system 100, for example as a tapered tip. Also, the distal tip 240 may comprise soft materials. Thus formed, distal tip 240 may be useful for navigating bodily lumens without causing trauma to the same.

In addition, any of the members may have a varying stiffness at any portion along its length to improve the performance of system 100. For instance, the inner member 120 may be comprised of any variety of physical materials that can flex according to the practitioner's needs. In some embodiments, the inner member 120 may have a plastic (synthetic) or a polymer (which may even include natural materials) core. Also, the proximal end 117 of the inner member 120 may comprise a relatively stiff portion 120″ in relation to the distal portion 119. The stiff portion 120″ of the inner member 120 may secure to the proximal handle 130.

Any of the members may have any number of varying diameters along its length provided that the variations serve to improve control of stent deployment of loading. For instance, the inner member 120 may include a distal flexible thick portion 120′ extending from and having a larger diameter than the proximal portion 120″ such that the proximal portion 120″ may be slidably disposed within the intermediate handle 150 while the distal portion 120′ may be slidable through only a portion of the intermediate handle 150. In such a case, the intermediate handle 150 may have a distal opening 420 larger than a proximal opening 440. Such arrangement may function as a stop or limit for the axial movement of the distal portion 120′ relative to the intermediate member 140 during the loading of the stent 300. The distal portion 120′ may be relatively flexible radially. This may provide limited flexibility so that the catheter system 100 can readily be pushed from the handle 130 through the patient's vessel with little risk of kinking. The flexible distal portion 120′ may be configured to support a compacted stent within the external member 160 proximate the distal end tip 240 for deployment from the system 100 in an expanded form within a patient's vessel.

The movement between members may be improved by reducing surface area contact, for example by having raised areas upon which an outer member would contact an inner member. Such raised areas or reduced surface areas may include bumps, ribs, etc.

The tubular members 120, 140, and 160 may be formed of a body compatible material such as a biocompatible polymer. In some embodiments, the stiff portion 120″ of the core inner member 120, which extends along a substantial length of the catheter system 100, may be formed of a plastic material such as PEX (cross-linked polyethylene) or an elongated coiled spring formed of plastic or metal such as Nitinol. In addition, the external member 160, which functions as a slippery outer sheath, may in some embodiments be formed of a radially flexible (bend sideways), axially stiff (not stretchable lengthwise) material such as polytetrafluorethylene (PTFE) or other like material as shall be described below.

The present invention, however, is not limited to the use of just PTFE as the external member, and other materials, including combinations of materials, may suitably be used. Specifically, any of the materials may be reinforced or improved by addition of reinforcement materials such as braids, coils or the like and/or geometric features and/or localized addition or subtraction of materials. For example, non-limiting examples of suitable biocompatible polymers also include, and are not limited to, polyolefins such as polyethylene (PE), high density polyethylene (HDPE) and polypropylene (PP), polyolefin copolymers and terpolymers, polyethylene terephthalate (PET), polyesters, polyamides, polyurethanes, polyurethaneureas, polypropylene and, polycarbonates, polyvinyl acetate, thermoplastic elastomers including polyether-polyester block copolymers and polyamide/polyether/polyesters elastomers, polyvinyl chloride, polystyrene, polyacrylate, polymethacrylate, polyacrylonitrile, polyacrylamide, silicone resins, combinations and copolymers thereof, and the like. Materials for the tubular members 120, 140, 160 may be same or different.

The tubular members 120, 140, and 160 may also have a surface treatment and/or coating on their inner surface, outer surface or portions thereof. A coating need not be applied to all of the tubular members 120, 140, 160, and individual members may be coated, uncoated, partially coated, and the like. Useful coating materials may include any suitable biocompatible coating. Non-limiting examples of suitable coatings include polytetrafluoroethylene, silicone, hydrophilic materials, hydrogels, and the like. Useful hydrophilic coating materials include, but are not limited to, alkylene glycols, alkoxy polyalkylene glycols such as methoxypolyethylene oxide, polyoxyalkylene glycols such as polyethylene oxide, polyethylene oxide/polypropylene oxide copolymers, polyalkylene oxide-modified polydimethylsiloxanes, polyphosphazenes, poly(2-ethyl-2-oxazoline), homopolymers and copolymers of (meth) acrylic acid, poly(acrylic acid), copolymers of maleic anhydride including copolymers of methylvinyl ether and maleic acid, pyrrolidones including poly(vinylpyrrolidone) homopolymers and copolymers of vinyl pyrrolidone, poly(vinylsulfonic acid), acryl amides including poly(N-alkylacrylamide), poly(vinyl alcohol), poly(ethyleneimine), polyamides, poly(carboxylic acids), methyl cellulose, carboxymethylcellulose, hydroxypropyl cellulose, polyvinylsulfonic acid, water soluble nylons, heparin, dextran, modified dextran, hydroxylated chitin, chondroitin sulphate, lecithin, hyaluranon, combinations and copolymers thereof, and the like. Non-limiting examples of suitable hydrogel coatings include polyethylene oxide and its copolymers, polyvinylpyrrolidone and its derivatives; hydroxyethylacrylates or hydroxyethyl(meth)acrylates; polyacrylic acids; polyacrylamides; polyethylene maleic anhydride, combinations and copolymers thereof, and the like. Additional details of suitable coating materials and methods of coating medical devices with the same among other features may be found in U.S. Pat. Nos. 6,447,835 and 6,890,348, the contents of which are incorporated herein by reference. Such coatings and/or surface treatment can be desirably disposed on the inside or a portion thereof of the external member 160 to aid, if desired, in loading and/or deploying of the stent.

Referring back to FIG. 1, the distal end tip 240 of the catheter system 100 may be fabricated to provide sideways mobility. That is, the distal end tip 240 remains free to float in radial (sideways) directions different from the axial direction of the core inner member 120. Thus, even a slight force directed upon the distal end tip 240 may nudge its position away from the source of the applied force. This feature can ease the practitioner's task in navigating the catheter system 100 through severely tortuous paths often associated with procedures in a patient's body, reducing steps and time necessary for delivering a stent to a delivery site.

Further as depicted in FIGS. 19 and 20, in some embodiments, the system 100 may include a stent basket 200 having opposed proximal and distal ends 202, 204 for engaging a stent 300. For instance, the proximal engaging end 202 may be securely disposed to or provided at the distal end 139 of the stent-loading intermediate member 140. The stent basket 200 may have a truncated-conical shape, being smaller at its proximal end, i.e., outwardly diverging in a distal direction from its proximal engaging end. The stent basket 200 may be a thin film which can collapse such that the stent basket 200 may be slidably contained within the distal end of the external member 160. Alternatively, the stent basket 200 may include a radially distensible member 200 as depicted in FIG. 20 which can be collapsible such that the stent basket 200 can be slidably contained within the external member 160. For instance, the stent basket may be a porous tube, a flexible tube, or any other configurable tube. In some embodiments, the stent basket 200 may be a polymeric member 200. The stent basket 200 may include, in part or substantially, braided filaments 206. The braided filaments 206 may include polymeric filaments, metallic filaments and any other suitable filaments. Alternatively, the braided filaments may be contained within a thin polymeric film.

Another preferred feature of the present invention is that a stent holder 210 can be provided on a distal portion 119 of the inner member 120 to temporarily hold the stent in place without any substantial external force acting on it. The stent holder 210 may be further defined by a tubular band 220. In some embodiments, the stent holder 210 may releasably hold stent 300 within system 100 even after the stent basket 200 may be axially displaced away from the stent 300. Such feature may allow, if desired, for a large portion of the stent 300 to be deployed and then be recaptured or re-engaged by stent basket 200 prior to complete deployment of the stent 300. The recapturing may be achieved by axially sliding the external member 160 over the stent 300. Moreover, the stent basket 200 may be repositioned between the inner member 120 and the external member 160, for example, by axially advancing the stent basket 200 to reposition the stent 300 therein between. Furthermore, the whole system 100 may be moved proximally or distally to reposition the stent 300 therein.

These features may provide, among other things, reloading ability (reconstrainability) of the stent 300 within the system 100 of the present invention. For example, the external member 160 may be advanced over the stent 300 to a location distally past the tubular band 220 to releasably and securely set the position of the stent basket 200 and/or the intermediate member 140 relative to the position of the inner member 120. The external member 160 may be retracted proximally past the tubular band 220, thereby allowing repositioning of the stent 300 within external member 160 and/or over the inner member 120. The external member 160 may be re-advanced over the stent 300 and the tubular band 220 to releasably and securely reset the position of the stent basket 200 and/or the intermediate member 140 relative to the position of the inner member 120, thereby reloading the stent.

The discrete members 120, 140, and 160 of the catheter system 100 facilitate flexibility and control. For instance, while the proximal and intermediate handles 130, 150 are fixed or relatively fixed, the practitioner may axially move distal handle 170 to cause the distal end 159 of the external member 160 to slide substantially up to the distal end 119 of the inner member 120. The independent or substantially independent sliding movement of the external member 160 is independent or substantially independent of the inner member 120 and the intermediate member 140 remain fixed or relatively fixed (for instance, to hold the stent).

In some embodiments, all three members, 120, 140 and 160 may be interlocked for initial deployment, repositioning and removal. Optionally, the proximal handle 130 may be axially moved between distal and proximal positions, while keeping the other handles 170, 150 fixed or relatively fixed. This axial movement facilitates independent or substantially independent movement of the inner member 120 while the external member 160 and the intermediate member 140 remain fixed or relatively fixed.

The present invention is not limited to those movements. In essence, the invention provides for axial movement of one particular member while the two other members are fixed relative to each other. Thus, the present invention includes any combined movement of the members 120, 140, and 160. For instance, any two of the handles 130, 150 and 170 may be moved or manipulated in concert as a pair while keeping the third or non-paired handle fixed or relatively fixed. This manipulation by the practitioner allows concurrent movement of two tubular members while keeping the third tubular member fixed or relatively fixed.

For example, the external member 160 and the intermediate member 140 may be moved in concert while keeping the inner member 120 fixed or relatively fixed. This movement can be achieved when the practitioner manipulates the distal handle 170 and the intermediate handle 150 in concert (in contrast to the movement of the proximal handle 130 above), while keeping the proximal handle 130 fixed or relatively fixed. Alternatively, the external member 160 and the inner member 120 may be moved in concert while the intermediate member 140 may be kept fixed or relatively fixed. This movement can be achieved by manipulating the distal handle 170 and the proximal handle 130 in concert while keeping the intermediate handle 150 fixed or relatively fixed. Moreover, the inner member 120 and the intermediate member 140 may be moved in concert while keeping the external member 160 fixed or relatively fixed. Similarly, this movement can be achieved by manipulating the proximal handle 130 and in concert with the intermediate handle 150 while the distal handle 170 is kept fixed or relatively fixed. It should be noted that a similar effect may be achieved by manipulating the distal handle 170 while the proximal handle 130 and the intermediate handle 150 are kept fixed or relatively fixed.

As described above, the stent basket 200 may be useful for engaging and securing a proximal end of a stent 300 and compressingly loading the stent 300 into the system 100 through axial manipulation. Such loading may be accomplished by gliding the intermediate handle 150, against the external member 160 and kept in that position until the desired stent delivery location within a bodily lumen (not shown) is reached. FIG. 1 also shows a stent holder 210 disposed on the inner member 120 at a distal location away from the stent basket 200. The system 100 may be configured to provide this stent holder 210 at the distal location when the intermediate handle 150 is proximally placed away from the distal handle 170. The system 100 may further advantageously include a tubular band 220 disposed toward the distal end of the inner member 120. Such tubular band 220 may releasably secure the stent 300 in the stent deployment region 110 between the inner and external members 120, 160.

Another feature of the present invention is that the stent holder 210 may be distally spaced apart from the stent basket 200. Such axial displacement (away from the stent basket 200) allows the stent holder 210 to releasably hold the stent 300 within the system 100 even after the stent basket 200 is withdrawn from the stent 300. Such feature facilitates, if desired, for a large portion of the stent 300 to be deployed and then be recaptured by the system 100 prior to complete deployment of the stent 300. Such recapturing or reconstrainability may be achieved with the filament described below or by axially sliding the external member 160 over the stent 300. Moreover, the stent basket 200 may be repositioned within the inner member 120 and the external member 160. For instance, the practitioner may axially advance the member 200 to reposition the stent 300 therein between. Furthermore, the whole system 100 may be moved proximally or distally to reposition the stent 300 therein in a body lumen.

Although the present invention is not so limited, the reconstrainability feature of the invention can be configured as follows. A practitioner may advance the external member 160 over the stent 300 to a location distally past the tubular band 220. The external member 160 advancement over the stent 300 releasably secures the stent basket 200 and/or the intermediate member 140 relative to the position of the inner member 120 to hold the stent 300 in place. When the stent is placed in the body lumen, the external member 160 may be retracted proximally past the tubular band 220, thereby allowing repositioning of the stent 300 within the external member 160 and/or over the inner member 120. If the practitioner decides to deploy the stent at a different location, the practitioner may re-advance the external member 160 over the stent 300 and the tubular band 220. This re-constraining releasably and securely reset the position of the stent basket 200 and/or the intermediate member 140 relative to the position of the inner member 120.

Although the current invention is not so limited, and the system 100 may be suited for delivery of many intraluminary devices, it may be particularly useful in loading and releasing the stent 300 such as the embodiment shown in FIG. 2. FIG. 2 illustrates a radially self-expanding stent 300 that can be radially compressed and loaded into system 100. This stent 300 may be transluminally delivered to an intended intraluminal treatment site, then released from the system for radial self-expansion against surrounding tissue of a bodily lumen. The degree of elongation may depend upon the structure and materials of the stent 300 and may be quite varied. The diameter of the stent 300 also may become several times smaller as it elongates. The radially distensible stent 300 may be a polymeric stent, including a braided stent. A graft, such as a covering, a liner, a film, a coating and combinations thereof, may be disposed over at least a portion of the stent. In some embodiments, the stent 300 may be a braided polymeric stent and the graft may be a silicone coating or film.

FIG. 2 also depicts the stent basket 200 details for the system 100 of the present invention. As depicted, the handles 150 and 170 can be disposed relatively towards one another such that the stent basket 200 may be exposed. When the stent basket 200 is exposed, it may include a distal portion radially extended to a diameter, for example, substantially larger by at least about double the diameter, than the outside diameter of the external member 160. The stent basket 200, which is depicted as being in the shape of a funnel or a basket, may be bonded, crimped or otherwise secured to the distal end of the intermediate member 140. In some embodiments, the stent basket 200 may have a truncated-conical shape, outwardly diverging in the distal direction from its proximal end, e.g., the proximal end being smaller than the distal end. The proximal end of the stent basket 200 may have a diameter equal or substantially equal, including slightly larger, to the diameter of the intermediate member 140, but less than the diameter of the external member 160.

The stent basket 200 may be formed of a thin polymeric film, for example, but not limited to, polyamide, such as polyamide 6-6 or nylon, PET or PTFE. In some embodiments, the film may be compliant, so that the funnel can alternatively assume an open configuration as seen in FIG. 1 for receiving a proximal end of stent 300. The film may also assume a collapsed configuration to allow the stent basket 200 to be accommodated or contained within external member 160. In some embodiments, the stent basket 200 may be resilient and can assume the open configuration in the relaxed state when free of external stresses. Alternatively, the stent basket 200 may be pliable, in particular radially distensible, mesh, weave or braid. The stent basket 200 may be of any reasonable length and/or diameter to permit the loading of the stent 300. The stent basket 200 may have a beveled edge or profile for easier loading, removing or repositioning of the stent 300. Further, the stent basket 200 may only partially circumferentially surround or encompass the intermediate member 140. Still further, the stent basket 200 may be split or slit at either or both of its distal and proximal ends. Moreover, the stent basket 200 may comprise a film with pores, thin spots or cut-outs.

As depicted in FIG. 13, in another embodiment, the distal and proximal openings 420, 440 of the intermediate handle 150 may be the same, substantially the same or about the same. In such embodiments, the intermediate handle 150 may be temporarily held against or near the distal handle 150 during loading of the stent 300 into the system 100.

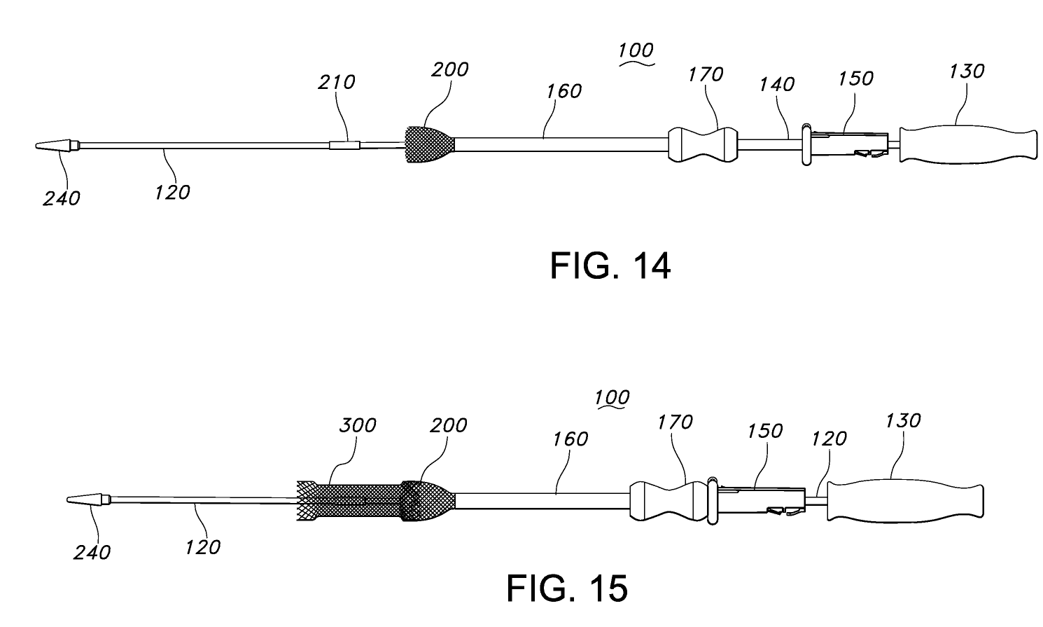

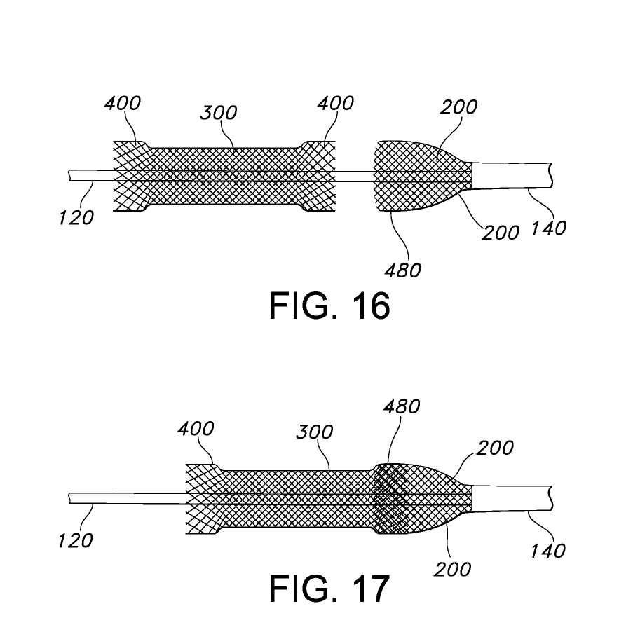

FIGS. 14 and 15 depict top planar views of the system 100 of the present invention. In this particular embodiment, the stent basket 200 may be a radially distensible basket, which can be made of similar materials or different materials from the material of the stent 300. As depicted in FIGS. 16 and 17, the stent basket 200 may have a truncated-conical shape 460, outwardly diverging in the distal direction from its proximal end. This shape may then merge, desirably seamlessly, into a straight or substantially straight cylindrical portion or rim portion 480. The stent basket 200 may be radially distensible, i.e., it may assume an enlarged state when released from a contracted state, such as being compressed within the external member 160. The stent basket 200 can be especially useful for engaging the stent 300 having an outwardly extending end.

As depicted in FIG. 19, the stent basket 200 may be made radially distensible with a truncated-conical shape by compressing a proximal portion of cylindrical stent basket 200 onto the intermediate member 140. Additionally, the rim portion 480 of the stent basket 200 may be inwardly biased, as depicted in FIG. 20. Such alternate stent engaging designs can be useful with the different stent configurations described herein. In some embodiments, the stent basket 200 may be secured to intermediate member as well.

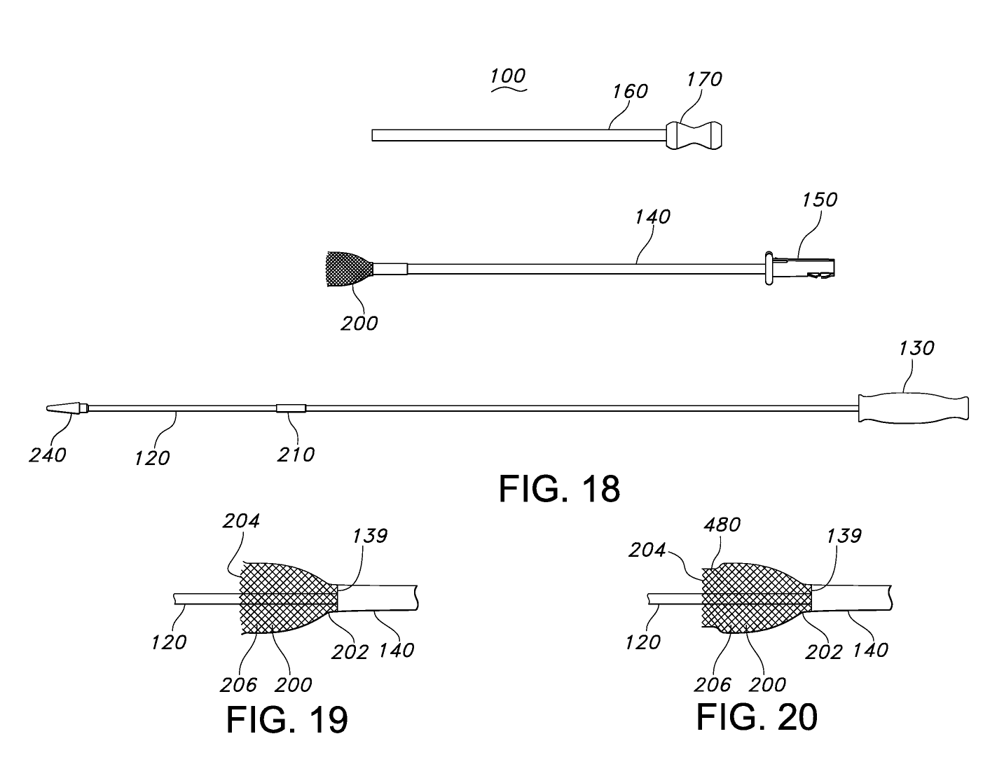

FIG. 18 is a top planar view of the different elements of the system 100 of the present invention in an “unassembled” stage. The inner member 120 can be the longest member. The intermediate member 140 may be smaller than the inner member 120, but longer than the external member 160. Finally the external member 160 may typically be the shortest of the members. The present invention, however, is not so limited and other tube length configurations may suitably be selected.

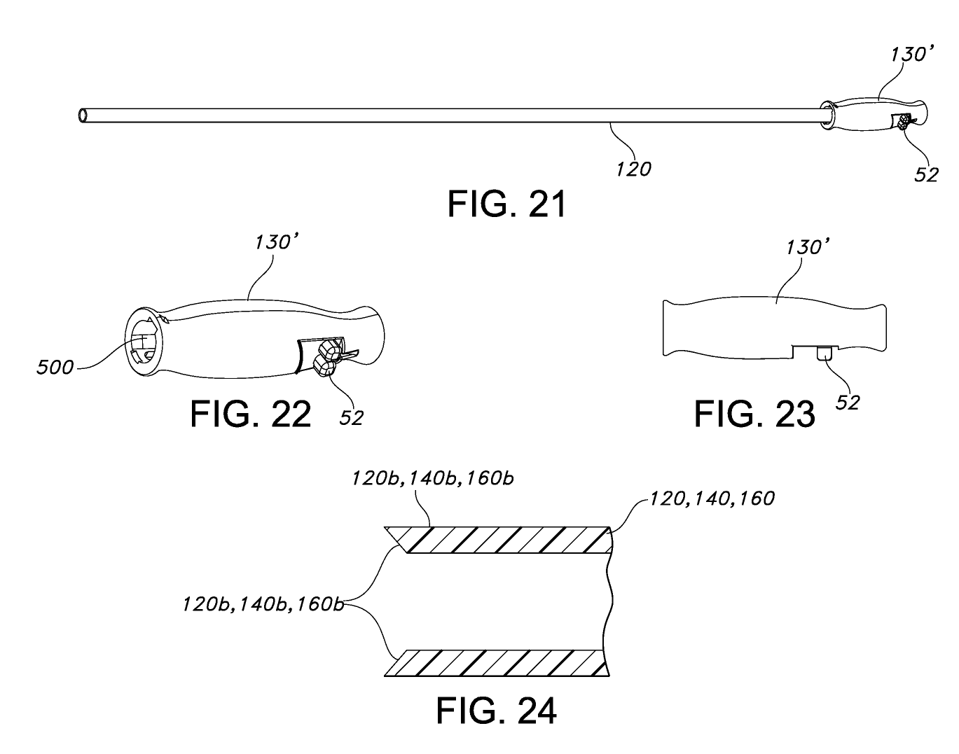

In the alternative, a central lumen can extend through the catheter system 100 and receive a guide wire. With the central lumen, the practitioner can first position the guide wire in the patient and then slide the catheter system 100 over the guide wire to position the catheter system 100 within the patient's vessels with relative ease. Moreover, the inner member 120 may be modified to enhance repositioning and/or retrieval of the stent 300. For example, as depicted in FIGS. 21 to 23, the proximal handle 130′ may include prongs 52. Prongs 52 are useful for securing a filament (not shown) to the outside of the handle 130′. The filament (not shown) may then be disposed within the cavity or lumen 500 of the handle 130. The filament may then exit at an intermediate point whereby the filament may be secured to the stent 300. The filament may be manipulated by the practitioner to reposition the stent 300 during or after its delivery. Upon completion of the stent delivery, the filament may be removed, for example by cutting, from the stent 300.

Such additional features are further described in U.S. application Ser. No. 11/437,455, entitled “Apparatus and Method for Loading and Delivering a Stent” (U.S. Pub. No. 2007/0270937 A1), filed on May 19, 2006, attorney docket 792-36, U.S. application Ser. No. 11/437,459, entitled “Apparatus and Method for Loading and Delivering a Stent Using a Suture Retaining Mechanism” (U.S. Pub. No. 2007/0270931 A1), filed on May 19, 2006, attorney docket 792-44, and, U.S. application Ser. No. 11/437,889, entitled “Apparatus and Method for Loading and Delivering a Stent” (U.S. Pub. No. 2007/0270932 A1), filed on May 19, 2006, attorney docket 792-57, the contents of which are incorporated herein by reference.

Further, the tubular members 120, 140, and 160, may have a beveled or slanted edge at their distal end, proximal end or combinations thereof. In some embodiments, such beveled edges may have smooth or round edges and accordingly may include rounded or smoothly contoured portions. For instance, as depicted in FIG. 24, tubular members 120, 140, 160, may have an inwardly beveled edge 120a, 140a, 160a at their respective distal ends 120b, 140b, 160b. In some embodiments, the beveled edge 160a can be an inwardly beveled edge on the distal end 160b of the external member 160. Such beveled edges, in particular, beveled edge 160a, are useful in aiding the loading and/or deployment of the stent 300. As depicted in FIG. 24, an inwardly beveled edge or end can be defined as a location where the wall of the tubular member has a greater longitudinal expanse at its outer wall portion as compared to its inner wall portion. In other embodiments, the edge may be rounded as well.

In addition, in some embodiments, the intermediate member 140 including the intermediate handle 150, and optionally the stent basket 200, may be releasably disposed to be completely removable from the device or system 100. For example, after loading the stent 300 into the system 100, the intermediate member 140 may be pulled proximally and completely detached from between the inner and external members 120, 160. Thus, although other methods exist, the intermediate member 140 may be split and pulled away from the inner member 120.

Another aspect of the invention improves the fluoroscopic observation of the distal end of system 100. Specifically, the distal end tip 240 can be formed of a radiopaque material or provided with a radiopaque marker. In addition, the core tubular member 120 and/or the extreme distal end of the tubular member 140 can also be provided with indicia material/marker and may be MRI compatible or of a type only visually perceived (e.g. colored baskets). Further the material of the system may be metal free as well.

Thus, the stent delivery system 100 of this invention can enable a practitioner to position a compacted stent at a selected location with full control. The practitioner may, with optional aid of selected indicia colors and interlocking basket 200, partially deploy the stent. Then, the practitioner may, upon observing a problem such as incorrect positioning or the like, either fully deploy the stent 300 or return the stent 300 to its compacted form within the basket 200.

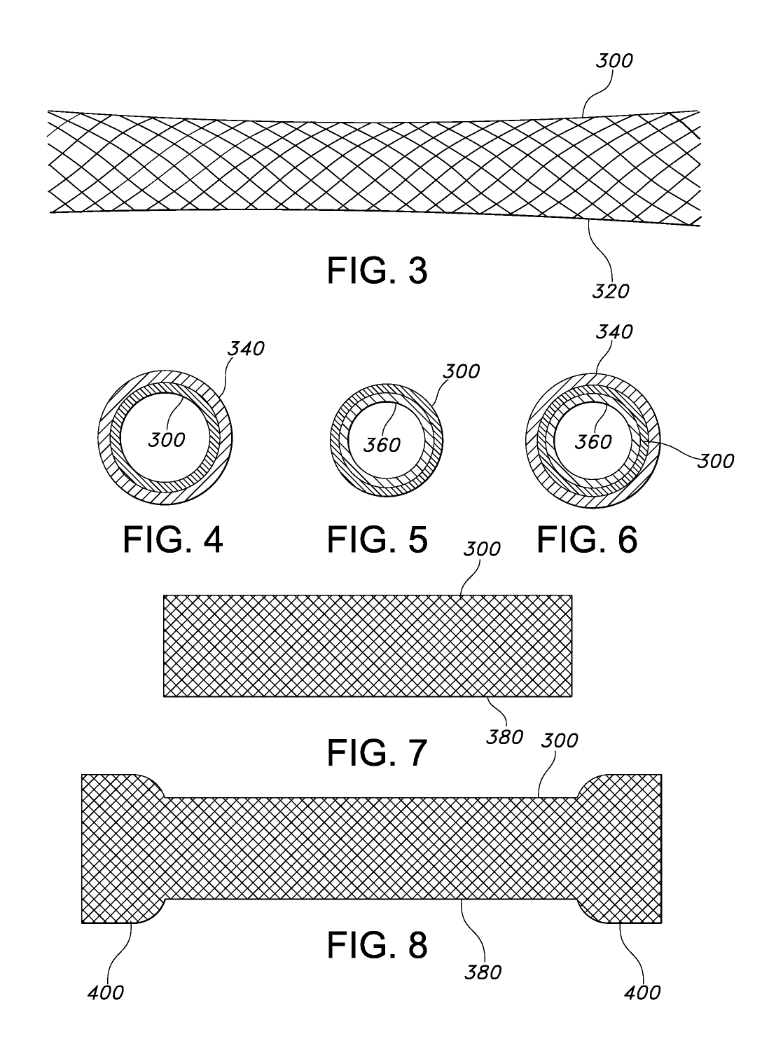

Any stent may be used in accordance with the present invention, and the invention can be constructed to accommodate stents of various sizes and configurations. For example, a radially distensible stent which does not substantially longitudinally elongate upon radial contraction is also useful. A non-limiting example of such a stent may be one formed from zig-zag or undulating wire or wires. Depicting a non-limiting example, FIG. 3 discloses an exploded or enlarged view of a braided stent 300 with braiding of the stent filaments 320. As used herein, the term braiding and its variants refer to the diagonal intersection of elongate filaments 320 so that each filament passes alternately over and under one or more of the other filaments, which is commonly referred to as an intersection repeat pattern. Useful braiding patterns include, but are not limited to, a diamond braid having a 1/1 intersection repeat pattern, a regular braid having a 2/2 intersection repeat pattern or a Hercules braid having a 3/3 intersection repeat pattern. The passing of the filaments under and over one and the other results in slidable filament crossings that are not inter-looped or otherwise mechanically engaged or constrained. The present invention is not limited by the above embodiment. For example, some or all of the filaments may be twisted at the filament or wire crossings. Ends of stent 300 may be atraumatic.

While the stent 300 may be formed of metals, plastics or any other materials, it is preferred that a biocompatible material or construction is employed. Useful biocompatible materials include, but are not limited to, biocompatible metals, biocompatible alloys, biocompatible polymeric materials, including synthetic biocompatible polymeric materials and bioabsorbable or biodegradable polymeric materials, materials made from or derived from natural sources and combinations thereof. Useful biocompatible metals or alloys include, but are not limited to, Nitinol, stainless steel, cobalt-based alloy such as Elgiloy, platinum, gold, titanium, tantalum, niobium, polymeric materials and combinations thereof. Useful synthetic biocompatible polymeric materials include, but are not limited to, polyesters, including polyethylene terephthalate (PET) polyester filaments, polypropylenes, polyethylenes, polyurethanes, polyolefins, polyvinyls, polymethylacetates, polyamides, naphthalane dicarboxylene derivatives, silks and polytetrafluoroethylenes. The polymeric materials may further include a metallic, a glass, ceramic or carbon constituent or fiber. Useful and nonlimiting examples of bioabsorbable or biodegradable polymeric materials include poly(L-lactide) (PLLA), poly(D,L-lactide) (PLA), poly(glycolide) (PGA), poly(L-lactide-co-D,L-lactide) (PLLA/PLA), poly(L-lactide-co-glycolide) (PLLA/PGA), poly(D,L-lactide-co-glycolide) (PLA/PGA), poly(glycolide-co-trimethylene carbonate) (PGA/PTMC), polydioxanone (PDS), Polycaprolactone (PCL), polyhydroxybutyrate (PlIBT), poly(phosphazene) poly(D,L-lactide-co-caprolactone) P LA/PC L), poly(glycolide-co-caprolactone) (PGA/PCL), poly(phosphate ester) and the like. Further, the stent 300 may include materials made from or derived from natural sources, such as, but not limited to collagen, elastin, glycosaminoglycan, fibronectin and laminin, keratin, alginate, combinations thereof and the like. Further, in some applications, the stent 300 may desirably be embedded in a coating of silicone. Additional details of such desirable stents are described in U.S. Pat. No. 6,162,244, the contents of which are incorporated herein by reference.

Further, the stent 300 may be made from polymeric materials which may also include radiopaque materials/markers to improve external imaging under magnetic resonance imaging (MRI) and/or ultrasonic visualization techniques. In essence, the stent 300 may be selectively made radiopaque at desired areas along the stent or made be fully radiopaque, depending on the desired end-product and application. Such radiopaque materials include complex metallic alloys, metallic-based powders or ceramic-based powders, particulates or pastes which may be incorporated into the polymeric material. For example, the radiopaque material may be blended with the polymer composition from which the polymeric wire is formed, and subsequently fashioned into the stent as described herein. Specifically, materials for enhancing MRI visibility include, but are not limited to, metal particles of gadolinium, iron, cobalt, nickel, dysprosium, dysprosium oxide, tantalum, gold, iridium, platinum, palladium, cobalt based alloys, iron based alloys, stainless steels, or other paramagnetic or ferromagnetic metals, gadolinium salts, gadolinium complexes, gadopentetate dimeglumine, compounds of copper, nickel, manganese, chromium, dysprosium and gadolinium. The stent 300 may have an inner core of tantalum, gold, platinum, iridium or combination of thereof and an outer member or layer of Nitinol to provide a composite filament for improved radiopacity or visibility. Alternatively, the radiopaque material may be applied to the surface of the metal or polymer stent. Various radiopaque materials and their salts and derivatives may be used including, without limitation, bismuth, barium and its salts such as barium sulfate, tantalum, tungsten, gold, platinum and titanium, to name a few. Additional useful radiopaque materials and other features may be found in U.S. Pat. No. 6,626,936, which is incorporated herein by reference.

Also, the stent 300 may have coverings, films, coatings, and the like disposed over, under or throughout or embedding the stent 300. For example, as depicted in FIG. 4, the stent 300 may include a covering 340. In some embodiments a polymeric covering, disposed over the longitudinal length or a portion of the longitudinal length of the stent 300. Further, as depicted in FIG. 5, the stent 300 may include a liner 360, desirably a polymeric liner, disposed within the longitudinal length or a portion of the longitudinal length of the stent 300. Moreover, as depicted in FIG. 6, the stent 300 may include a both a covering 340 and a liner 360, desirably a polymeric covering and liner which include the same or different polymeric materials, disposed over and within the longitudinal length or a portion of the longitudinal length of the stent 300. Other features, which may be included with the stent 300 of the present invention, include surface modification for ultrasound, cell growth or therapeutic agent delivery; varying stiffness of the stent or stent components; varying geometry, such as tapering, flaring, bifurcation and the like; varying material; varying geometry of stent components, for example tapered stent filaments; and the like.

The covering and the liner of FIG. 6 may be a unitary film or coating that embeds or partially embeds the stent 300. The coating or coatings may be on the stent 300, components of the stent 300, and combinations thereof. The covering 340 and/or the liner 360 may be in the form of a tubular structure, for example composed of polymeric material and/or silicone. The covering 340 and/or the liner 360 may also comprise any plastic or polymeric material, desirably a somewhat hard but flexible plastic or polymeric material. The covering 340 and/or the liner 360 may be transparent or translucent, desirably substantially or partially transparent. The stent components, in part or in total, may be temporary, for example bio-absorbable, biodegradable, and the like, or may be permanent (i.e., not substantially bio-absorbable or biodegradable), for example the above-described biocompatible metals, alloys and polymers.

Furthermore, the covering 340 and/or the liner 360 may be constructed of any suitable biocompatible materials, such as, but not limited to, polymers and polymeric materials, including fillers such as metals, carbon fibers, glass fibers or ceramics. Useful covering 340 and/or the liner 360 materials include, but are not limited, polyethylene, polypropylene, polyvinyl chloride, polytetrafluoroethylene (PTFE), including expanded polytetrafluoroethylene (ePTFE), fluorinated ethylene propylene, fluorinated ethylene propylene, polyvinyl acetate, polystyrene, poly(ethylene terephthalate), naphthalene dicarboxylate derivatives, such as polyethylene naphthalate, polybutylene naphthalate, polytrimethylene naphthalate and trimethylenediol naphthalate, polyurethane, polyurea, silicone rubbers, polyamides, polyimides, polycarbonates, polyaldehydes, polyether ether ketone, natural rubbers, polyester copolymers, styrene-butadiene copolymers, polyethers, such as fully or partially halogenated polyethers, silicones, and copolymers and combinations thereof.

Further, the stent 300 may be treated with a therapeutic agent or agents, such as, but not limited to, anti-thrombogenic agents (such as heparin, heparin derivatives, urokinase, and PPack (dextrophenylalanine proline arginine chloromethylketone); anti-proliferative agents (such as enoxaprin, angiopeptin, or monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-miotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, antithrombin compounds, platelet receptor antagonists, anti-thrombin antibodies, anti-platelet receptor antibodies, aspirin, prostaglandin inhibitors, platelet inhibitors and tick antiplatelet peptides); vascular cell growth promotors (such as growth factor inhibitors, growth factor receptor antagonists, transcriptional activators, and translational promotors); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vascoactive mechanisms.

Further, as depicted in FIG. 7, the stent 300 according to the current invention may have a straight or substantially straight longitudinal portion 380. The present invention, however, is not so limited. For example, the stent 300 may have a varied diameter, such as a flaring or tapering, along a portion or portion of its longitudinal expanse. Further, the stent 300 may include anti-migration features such as flares, steps, ridges, anchors, fasteners or other protrusions on its external surface. One non-limiting example of a varied diameter stent 300 is depicted in FIG. 8. The stent 300 of FIG. 8 may include a longitudinal length 380 and one or two flared ends 400. As depicted in FIG. 8, the flared ends 400 may include enlarged flared ends having a diameter greater than the diameter of the longitudinal portion 380 of the stent 300. However, alternatively, the flared ends 400, individually or in combination, may have a smaller diameter than the diameter of the longitudinal portion 380 of the stent 300.

Additionally, the stent 300 itself may include anti-migration features such as flares, steps, ridges, anchors, fasteners, etc. Further, the stent 300 may be repositionable, removable and/or reconstrainable, and/or may include multiple interconnected or non-interconnected filaments. For example, the stent 300 may include a filament loop or element, such as a suture loop or element, a polymeric loop or element, metallic or element, and combinations thereof. Such additional loop or element may be accessible to a practitioner, for example by the use of forceps, to reposition, remove and/or reconstrain the stent 300 after it has been delivered, partially or totally, to a bodily lumen. Moreover, a loop or element may be integrally formed as part of the stent 300. Further details of useful repositioning, removing and/or reconstraining loops or elements may be found in U.S. patent application Ser. No. 11/341,540, filed Jan. 27, 2006 and entitled “Stent Retrieval Member And Devices And Methods For Retrieving Or Repositioning A Stent” (U.S. Pub. No. 2006/0190075 A1) and in U.S. patent application Ser. No. 11/432,065, filed May 11, 2006, attorney docket 792-32, and entitled “Integrated Stent Respostioning And Retrieval Loop” (U.S. Pub. No. 2006/0276887 A1), the contents of both of which are incorporated herein by reference.

The method of utilizing the system 100 is also contemplated by the present invention. In some embodiments, the utilization may include a method for loading, delivery and deployment of a stent 300 utilizing the system 100 in percutaneous, transluminal or other insertion techniques. The system 100 allows the practitioner to easily load a stent into a delivery system 100 with minimal effort and without damaging the stent 300.

In an initial setting, the stent delivery system 100 can be supplied to the practitioner in a protective package. The stent 300 in the package may be supplied in an unconstrained condition (so the plastic stent does not take a “set”). When ready to be used, the practitioner may take the delivery system 100 and the stent 300 out of the package in preparation of the procedure. As discussed above, a packaged delivery deployment system 100 typically may include an external member 160, an intermediate member 140, and an inner member 120, each having opposed distal and proximal ends with respective handles 170, 150, and 130. Each of these handles 170, 150, and 130 control the respective member's slidable disposition; optionally, a stent basket 200 may be securely disposed to the distal end of the stent loading intermediate member 140.

The handles 130, 150, and 170 may be supplied constrained in the package. Such constraint may be provided, for instance, by the way of friction or mechanical locks, to prevent the practitioner from accidentally moving the handles 130, 150 and 170. Out of the package, the shaft portion 155 of the intermediate handle 150 may be provided partly in the receptacle 134 of the proximal handle 130 through a mechanical and locking mechanism. In this position, the distal end 139 of the intermediate member 140 is extended away from the practitioner to an extended (distal) position 153. In such distal position 153, the stent basket 200 attached to the distal end 139 of the intermediate member 140 may be in an unconstrained position and readily accept a stent 300.

The practitioner may place the stent 300 over the stent holder 210 provided at distal end 139 of the intermediate member 140 as follows. First, the proximal end of the stent 300 may be placed within the stent basket 200, as depicted in FIG. 15. Afterwards, the stent 300 may be squeezed or radially contacted onto or about the inner member 120. The stent 300 may then be pushed into the stent basket 200 to facilitate its engagement within the intermediate member 140. Alternatively, the stent 300 may be disposed within a loading cartridge (not shown) for facilitating storage and delivery of the stent 300 into the intermediate member 140. The loading cartridge may contain a piston or other axially movable member to facilitate stent movement. Details of suitable stent loading cartridges are further described in U.S. Pat. No. 6,068,635 and/or U.S. Patent Application Publication 2003/0083730 A1, the contents of which are incorporated herein by reference.

During the loading of the stent 300, the handles 170 and 150 may be kept fixed in relatively constant axial displacement from one and another. As such, the inner member 120 and the intermediate member 140 may also be kept in relative constant axial positions with the intermediate member 140 being substantially disposed within the external member 160. However, the intermediate member 140 need not be completely contained within the external member 160. Rather, a portion of the distal end of the intermediate member 140 may be axially outside or distally disposed from the distal end of the external member 160. Additionally, the smaller distal opening or flange of the intermediate handle 150 may serve as a stop or an axially limiting mechanism. Such limiting mechanism keeps the inner and intermediate members 120, 140 in relative constant axial arrangement during loading of the stent 300. To complete the stent 300 loading, the proximal handle 130 may be pulled away from the distal handle 170 to complete the loading.

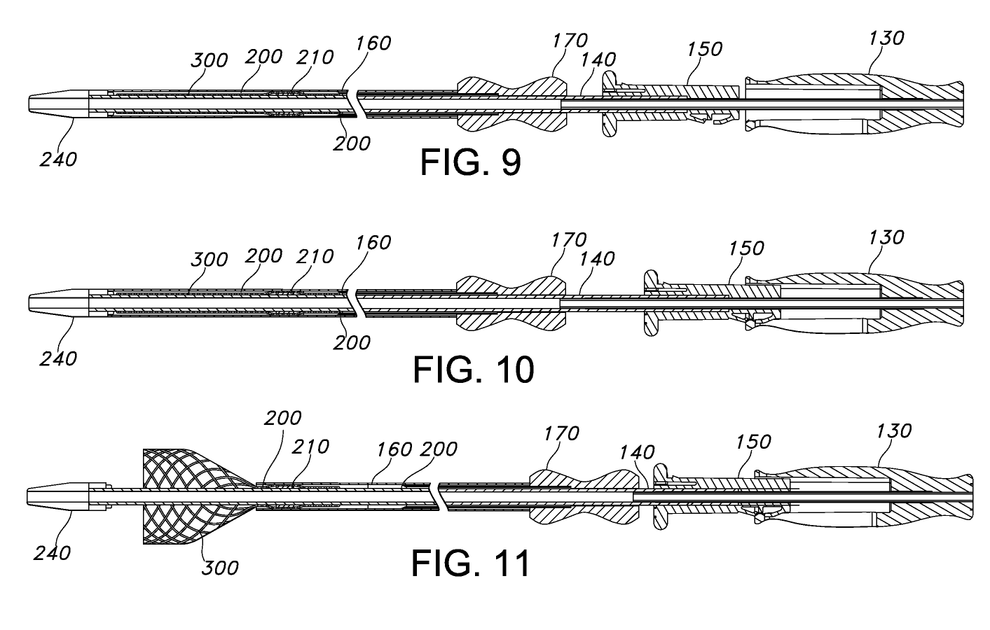

In some embodiments, the intermediate basket loading member may also be fully utilized. In the embodiment of this invention illustrated by FIGS. 9 and 10, the practitioner may load the stent 300 by sliding the distal handle 170 in a distal direction. This action can slide the external member 160 onto the stent 300 to load and position the stent 300 well into the distal end of the external member 160. The practitioner then may move the distal handle 170 further distally to constrain the stent 300 until the stent can be fully constrained by the external member 160. The external member 160, attached to the distal handle 170, can then slide over the stent basket 200 and collapse and constrain the stent 300 completely over the inner member 120.

The present invention may also include a method of providing the tubular band 220 disposed toward the distal end of the inner member 120. Such method may releasably secure the stent 300 in the stent deployment region 110 between the inner and members 120, 160. Moreover, the method may further include axially sliding the external member 160 toward a proximal position for releasing the stent 300 from the stent deployment region 110. The method may yet further include providing a distal handle 170 disposed at the proximal end 157 of the external member 160; providing a proximal handle 130 disposed at the proximal end 117 of the inner member 120; and providing a stent loading intermediate handle 150 disposed at the proximal end 137 of the intermediate member 140. The method of independently moving the external member 160, the inner member 120 or the intermediate member 140 may be achieved by manual manipulation of the handles 170, 130, 150.

The practitioner may manipulate the handles 130, 150, and 170 as follows: As depicted in FIG. 10, the practitioner may grasp the proximal handle 130 and the intermediate handle 150 with two hands. The practitioner may, then, utilize the intermediate handle lock and may actuate a release mechanism to release the intermediate handle 150 from the protrusion 136. This act retracts the intermediate handle 150 into the proximal handle 130. This retracts the intermediate member 140 relative to core inner member 120. The system 100 may be configured such that mechanism 154 can be overcome when retracting the basket 200. As a result, the stent 300 can be positioned within the external member 160 with the portions overlying the basket 200.

Thus formed, the practitioner may slide the distal handle 170 in the direction away from the intermediate handle 150 and the proximal handle 130. This can actuate the external member 160 to extend over the stent basket 200 along with the stent 300 lying therein which collapses into a constrained position inside the external member 160. Once the external member 160 completely engages the stent basket 200, the practitioner can pull back the intermediate handle 150 in relation to the proximal handle 130 and the distal handle 170. As the intermediate handle 150 is pulled back, the inner member 120 keeps the stent from traveling back as well. Thus, the stent basket 200 releases the stent 300 within the external member, which then retain the stent there within. Thus, the stent may be properly loaded into the system 100. Once the intermediate handle 150 is pulled back completely, the mechanical lock 136 and the protrusion 152 combination locks the position of the intermediate handle 150 in step with the proximal handle 130 so that the intermediate handle and the proximal handle now form a single handle. The delivery system can now be ready to be inserted into the body.

In some embodiments, the delivery system as depicted in FIG. 10, the stent basket 200 may be moved away from the constrained stent 300. Afterwards, the stent 300 may be completely loaded or contained within the external member 160, and the intermediate handle 150 may be advanced proximally away from the distal handle 170 and toward the proximal handle 130 until it locks into the proximal handle 130. This effectively moves the stent basket 200 axially away from the loaded stent 300. In other words, the proximal end of the loaded stent 300 can be now free from the stent basket 200. Such removal of the stent basket 200 from the loaded stent 300 facilitates the delivery of the stent 300 as less force may be required to deploy the stent 300. Also, this ensures that the stent basket 200 will not interfere with the stent 300 while the stent is being deployed. Now, only the distal handle 170 needs to be manipulated to deploy the stent 300. In some embodiments, the handles may be designed with a less accessible intermediate handle release mechanism (basket retraction handle). Alternatively, the practitioner can control the position of the intermediate handle by another feature such as a rod, which may interlock with the proximal handle 130. Also, the retention of the intermediate handle 150 can be made permanent by features such that the handles cannot be separated if this feature is desirable.

As depicted in FIG. 9 and as described above, the stent 300 can be fully loaded into the system 100 of the present invention. Thus positioned, the stent holder 210 releasably secures the stent 300 between the inner member 120 and the external member 160. In some embodiments, the stent holder 210 may be a hollow tubular band. More desirably, the stent holder 210 may be a hollow tubular band that may be free or substantially free of barbs, pins or protrusions which may engage and possible damage the stent 300. The stent holder 210 may be made of any suitable polymeric, rubber or metallic material. In some embodiments, the stent holder 210 may be a raised portion of the inner member 120. Moreover, the stent holder 210 may have adhesives or a pattern, such as a surface pattern of indentations and/or protrusions, for facilitating securement of the stent 300. In some embodiments, the stent holder 210 may have barbs, pins or protrusions which may engage the stent 300. Further, with any of the embodiments, the system 100 may include multiple stent holders 210, either axially spaced apart or axially juxtaposed. Further, the stent holder 210 may not have to completely encompass the inner member 120, but may be only partially disposed around a circumferential portion of the inner member 120.

The stent delivery system 100 can now be positioned in the patient and the stent 300 deployed by moving the distal handle 170 in a proximal direction. The intermediate handle 150 may have a release mechanism such that the intermediate handle 150 can be repositioned back to the original position if the stent 300 needs to be re-loaded. If needed, the stent 300 can be removed from the body and reloaded on the delivery system 100 by unlocking the intermediate handle and returning it to the distal position. Optionally, the system can be positioned by axially moving or sliding the stent engaging basket 200 to a location past the stent deployment region 110 for disengagement of the stent 300 from the intermediate member 140.

The practitioner can next perform the insertion of the distal end of the system 100 into a patient's body with a lead-in such as the distal end tip 240. Once the practitioner navigates the distal end tip 240 to a desired location, and is satisfied with the location and orientation of the partially deployed stent 300, the practitioner can actuate the handle 170 to fully deploy the stent 300 as depicted in FIG. 33. The practitioner can then pull back the distal handle 170 toward the intermediate handle 150, thereby pulling back the external member 160. This step uncovers the constrained stent 300 and may be designed to unload the stent into the desired deployment region. The delivery system 100 can then be removed from the body.

As depicted in FIG. 11, the loaded stent 300 may be unloaded to a bodily lumen (not shown) by advancing the distal handle 170 and correspondingly the external member 160 axially away from the distal tip 240. When the practitioner partially retracts the tubular member 160 to a position as shown in FIG. 25, the distal portion of the stent 300 beyond the sheath may expand. The practitioner can use fluoroscopy, MRI, endoscopy or other minimal invasive viewing techniques and apparatus to determine whether the stent 300 is appropriately positioned. Once the system 100 is properly inserted, the practitioner may position the distal end tip 240 at a selected location in a patient's vessel for deployment of the stent 300. This can be accomplished by a various means, in some embodiments by providing markers such as radiopaque rings or indicia on the core part proximate the distal end tip 240 to properly locate the system 100.

Some factors, which are only apparent upon deploying the stent, can render the location initially thought to be inappropriate, end up as the most optimal location. For these reasons, the embodiment of FIG. 1 may include indicia there on, so that a practitioner using any number of imaging methods such as fluoroscopy, MRI, ultrasound, etc. may ascertain the extent of deployment of the stent 300, as more fully discussed below. The practitioner may ascertain whether the stent 300 is properly positioned by any number of imaging methods before fully deploying the stent 300 and retract the partially deployed stent 300. This can be important to both the practitioner and the patient as improper positioning can increase the trauma and risk to a patient and curtail the efficiency of the treatment. If the stent 300 is not properly positioned, the practitioner may displace the handle portion 130 proximally to retract the core inner member 120 and the intermediate member 140 fully within the tubular member 160. Such displacement can ensure that the stent 300 returns to the condition as depicted in FIG. 10. The distal end can then be maneuvered to another desired location or into a desired orientation and the process repeated.

As depicted in FIGS. 10 and 11 and described above, the intermediate handle 150 and the proximal handle 130 may be proximally and/or juxtaposingly disposed during certain stages of loading, constraining and/or deploying the stent 300. Accordingly, the stent loading intermediate handle 150 may be integrated, for example mechanically integrated, with the proximal handle 130 to permit concurrent or simultaneous movement of the two handles 130, 150. Such mechanical integration, may be achieved by matching and/or interlocking mechanisms (FIG. 1) on the two handles 150, 130. The mechanical integration may be achieved through various means such as interference fit, adhesive fit, thread fit, soft material that compresses to fit the intermediate handle 150 into the proximal handle 130, any other equivalent fit thereof. In some embodiments, the use of releasably interlocking mechanisms (FIG. 1) on the two handles 130, 150 may provide the mechanism integration. This mechanism may permit independent movement of the two handles 130, 150 by mechanically releasing the mechanisms from one and another. It may also permit concurrent or simultaneous movement of the two handles 130, 150 by mechanically engaging the mechanisms with one and another. In some embodiments, the fit between the handles may be interference fit or could have a soft material that compresses to fit intermediate handle into proximate handle. Further, other embodiments may be adhesive, or threads, etc.

FIGS. 21 to 24 depict another embodiment of this invention. FIG. 22 shows an enlarged view of an embodiment of the proximal portion of the system 100 of the present invention. The handle interlock and the protrusion 52 may be a single-molded in the design of the intermediate handle. A step or cut-away portion of the inner member 120 may optionally serve as the above-described proximal portion. As described, such a proximal portion in conjunction with the small proximal opening 440 of the intermediate handle 150 serves as a stop during loading of the stent 300 into the system 100 of the present invention.

Another feature of the present invention is that the stent loading can be reversible. If the practitioner suspects that stent 300 was incorrectly positioned during loading, or determines that a different stent should be used, stent 300 can be easily unloaded, by operating handles 130 and 150 to advance inner member 120 toward the open position. This progressively releases stent 300 from the external member 160, whereupon the stent 300 may be removed from stent basket 200 by hand. Alternatively, if the practitioner needs to adjust the position of deployment or retract the stent, the practitioner may push the distal handle 170 away from the intermediate handle 150 to move the external member over the stent. Then, the practitioner is free to either move the stent to another deployment region or take it out completely. Further, the external member 160 may be retracted proximally past the tubular band 220, thereby allowing repositioning of the stent 300 within the external member 160 and/or over the inner member 120. The external member 160 may be re-advanced over the stent 300 and the tubular band 220 to releasably and securely reset the position of the stent basket 200 and/or the intermediate member 140 relative to the position of the inner member 120, thereby allowing reconstrainment of the stent.

The above described assembly may be suited for medical applications such as in the gastrointestinal tract, the biliary tract, the urinary tract, and the respiratory tract. The assembly in accordance with the present invention, however, could also be used in the neurological system (e.g., in the brain), the vascular system (e.g., in arteries or veins), in the cardiovascular system (e.g., in the heart) and in the like. Further, the assembly may be used in artificially created passageways as well. Reference to bodily passageways may be to passageways in any of the aforementioned tracts and systems or elsewhere in the body. In some embodiments, the assembly may be used in artificially created passageways.

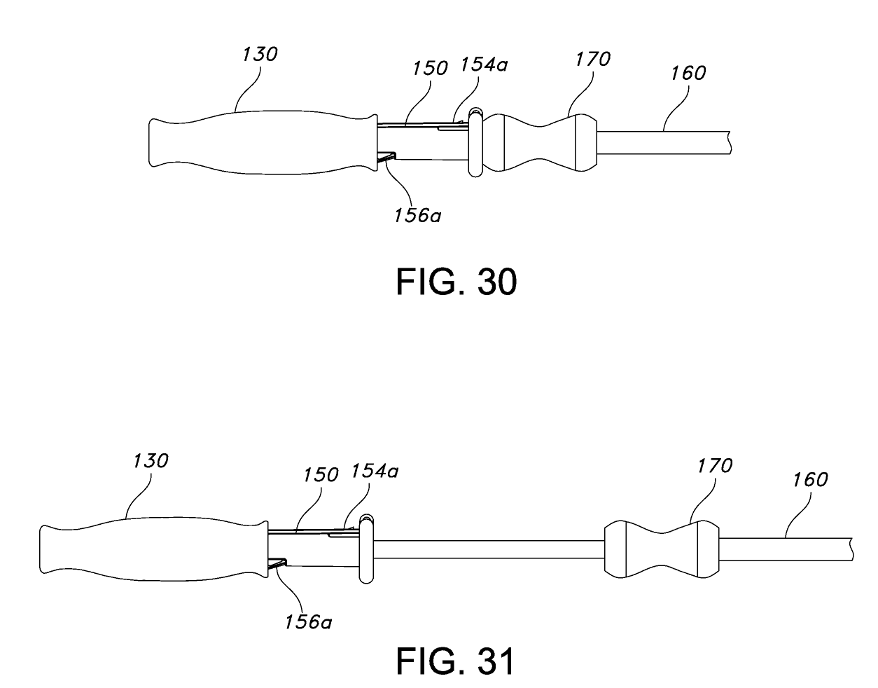

The stent delivery system 100 is typically supplied to the practitioner in a protective package (not shown). The stent 300 may be supplied in an unconstrained condition within the package or may be supplied separately from the package. As depicted in FIG. 30, intermediate handle 150 is in a distally extended position with respect to the proximal handle 130. With the intermediate handle 150 so distally extended, the stent basket 200 is also is a relative distal position such that it may accept the stent 300. As previously described, the handles 130, 150, 170 are designed so that in the as-packaged condition, their relative movement is restricted or minimized either by friction or mechanical locks.

As depicted in FIG. 31, after the stent 300 is loaded into the delivery system 100, the distal handle 170 is moved distally to constrain the stent 300 until the stent 300 is fully constrained by the outer catheter or outer exterior member 160.

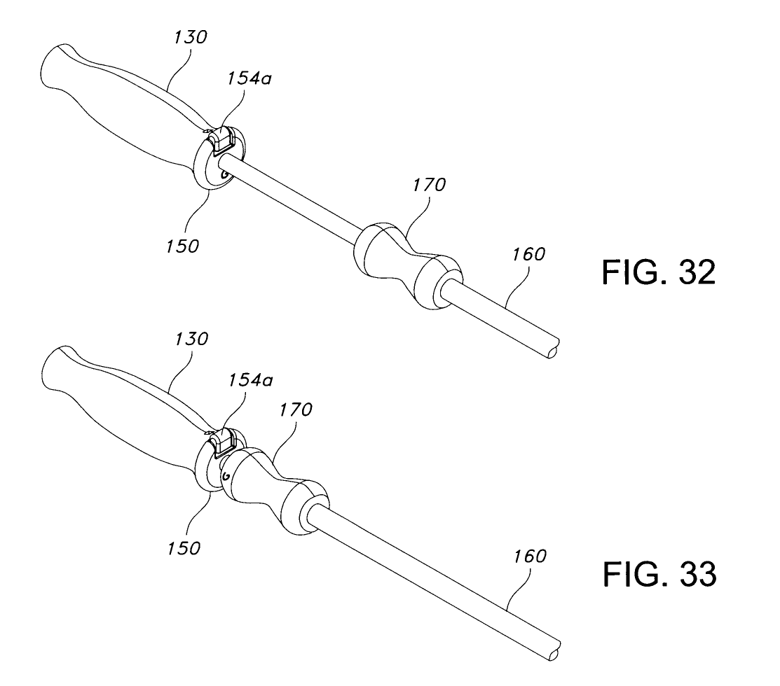

As depicted in FIG. 32, the stent basket 200 is then retracted by sliding the intermediate handle 170 in a proximal direction towards the proximal handle 130 until the intermediate handle 150 locks into the proximal handle 130 and the basket 200 is no longer in contact with the stent 300. After the intermediate handle 150 is locked into the proximal handle 130, advantageously there is now only one handle which may be manipulated to deploy the stent 300.

The stent delivery device 100 may then be positioned in a patient by a practitioner. As depicted in FIG. 33, the stent 300 may be deployed by a practitioner by moving the proximal; handle 130 in a proximal direction toward handles 130, 150. If needed, the stent 300 may be removed from the body or repositioned within the body by reloading the stent 300 onto the delivery device 100. Such reloading may be accomplished by unlocking the intermediate handle 150, for instance by actuating mechanism 154a, and moving the intermediate handle 150 distally away from the proximal handle 130.

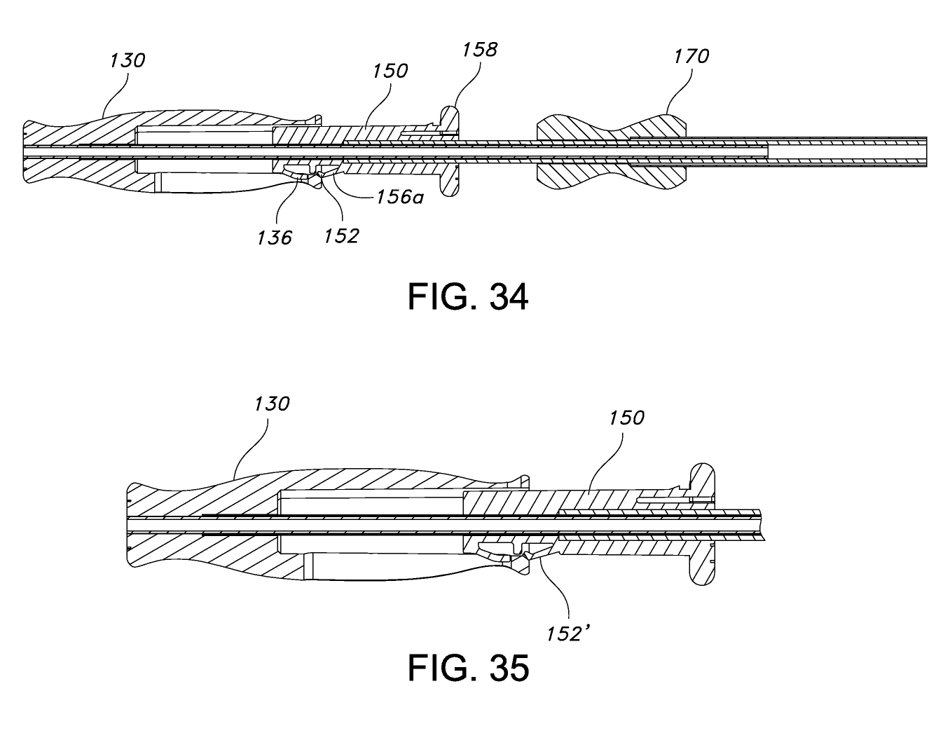

As depicted in FIG. 34, the handle interlock 152 prevents or inhibits unwanted movement of the intermediate handle 150 from moving too far distally away from the proximal handle 130. Further, the protrusion 136 and ledge 156a aids in the positioning of the stent basket 200 for proper stent placement during constraining of the stent 300 within the delivery system 100. As depicted in FIG. 25, the intermediate handle 150 is slidable into the proximal handle and may be releasably contained therein.

As depicted in FIG. 35, the handle interlock 152′ may be, if desired, a single molded feature of the intermediate handle 150.

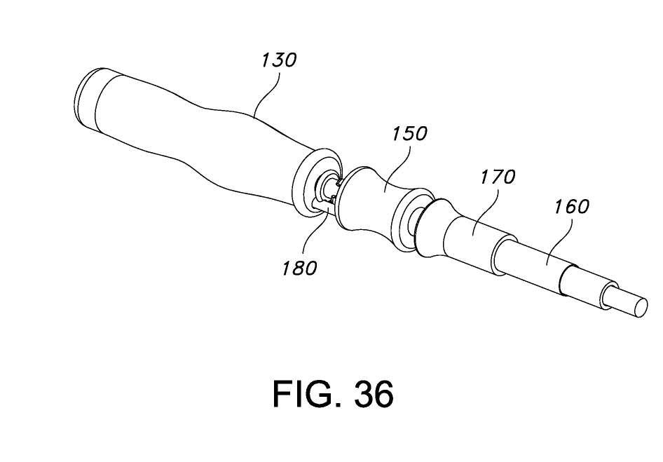

As depicted in FIG. 36, the positioning of the intermediate handle 150, which serves in part as a basket 200 retraction handle, my be controlled by another feature, such as a rod 180 which interlocks with the proximal handle 130. Such a rod 180 may also provide limited motion of the intermediate handle, as depicted in FIG. 26.

While select preferred embodiments of this invention have been illustrated and/or described herein, those skilled in the art will appreciate that many modifications and variations of the present invention may occur and therefore it is to be understood that these modifications are incorporated within these embodiments as if they were fully illustrated and described herein without departing from the spirit and intended scope of the invention. For instance, the features of this aspect of the present invention may suitably be combined in any combination according the present invention. In other words, all possible combinations of the features or elements of this aspect of the present invention are contemplated, including all features and elements described in conjunction with the drawings.

1. A delivery catheter handle assembly comprising:
a proximal handle attached to a proximal end of an axially elongated inner member;
an intermediate handle attached to a proximal end of an axially elongated intermediate member, at least one of said proximal and said intermediate handles being designed to accommodate and engage the other of said proximal and said intermediate handles; and
a distal handle attached to a proximal end of an axially elongated external member overlying at least a portion of said intermediate member.

2. The delivery catheter handle assembly according to claim 1, wherein said proximal handle and said intermediate handle include a handle-interlock mechanism, a portion of said intermediate handle being releasably retained by said proximal handle to prevent distal movement of said intermediate handle.

3. The delivery catheter handle assembly according to claim 2, wherein said handle-interlock mechanism is molded unitary with said proximal handle and said intermediate handle.

4. The delivery catheter handle assembly according to claim 1, further comprising:
a slide-and-lock mechanism, wherein one of said proximal handle and said intermediate handle is retracted into the other to define a unitary retracted position configured to limit travel between said proximate handle and said intermediate handle.

5. The delivery catheter handle assembly according to claim 4, wherein said intermediate handle includes a release mechanism such that the intermediate handle can be released and positioned relatively away from said proximal handle to permit re-loading of a prosthesis.

6. The delivery catheter handle assembly according to claim 4, wherein said slide-and-lock mechanism is molded unitarily with said proximal handle and said intermediate handle.

7. The delivery catheter handle assembly according to claim 1, wherein said intermediate handle includes a mechanism at a distal position configured to prevent unintended retraction.

8. The delivery catheter handle assembly according to claim 1, wherein said intermediate handle includes at least one projecting finger for interlockingly connecting with said proximal handle.

9. The delivery catheter handle assembly according to claim 1, wherein said intermediate handle includes a gripping surface.

10. The delivery catheter handle assembly of claim 9, wherein said gripping surface includes wings.

11. The delivery catheter handle assembly of claim 9, wherein said gripping surface includes a soft grip.

12. The delivery catheter handle assembly of claim 1, further including a guidewire port.

13. The delivery catheter handle assembly according to claim 1, wherein said proximal handle includes projections to provide frictional force retaining said intermediate handle during the prosthesis loading process.

14. The delivery catheter handle assembly according to claim 1, wherein said proximal handle includes a mechanism for interlockingly capturing said intermediate handle during the prosthesis loading process.

15. The delivery catheter handle assembly according to claim 1, wherein in an as-packaged condition, the relative movement of the handles is mechanically limited.

16. The delivery catheter of claim 1, wherein said intermediate handle further includes a locking mechanism for connecting with said proximal handle, said locking mechanism having a release mechanism which is recessed.

17. An apparatus for loading and delivering a prosthesis, the apparatus comprising:
a prosthesis;
a core segment including a proximal handle, an axially extended inner member formed integral with said proximal handle;
a middle segment including an intermediate handle, an axially elongated intermediate member formed integral with said intermediate handle, wherein one of said proximal and said intermediate handles are designed to assume and engage other of said proximal and said intermediate handles; and
an external segment including a distal handle, an axially elongated external member formed integral with said distal handle and overlying at least a portion of said intermediate member.

18. The apparatus according to claim 17, wherein said proximal handle and said intermediate handle include a handle-interlock mechanism, wherein a portion of said intermediate handle is releasably retained by said proximal handle.

19. A method for loading prosthesis onto a delivery system, the method comprising:
providing a prosthesis delivery system including a proximal handle attached to an inner member, an intermediate handle attached to an intermediate member, one of said proximal and said intermediate handles being designed to accommodate and engage another of said proximal and said intermediate handles, and a distal handle attached to an external member overlying at least a portion of said intermediate member, said proximal handle having a releasable locking mechanism for releasably engaging said intermediate handle;
providing a prosthesis in a unconstrained condition;
positioning said intermediate handle in a position away from said proximal handle and loading said prosthesis;
moving said distal handle distally to constrain said prosthesis within said external member; and
retracting said prosthesis basket into said axially elongated intermediate member by sliding said intermediate handle in a proximal direction until it locks into said proximal handle via said releasable locking mechanism and said basket is no longer in contact with said prosthesis.

20. The method for loading prosthesis according to claim 19, the method further including the steps of:
positioning said prosthesis delivery system into a patient; and
deploying said prosthesis by moving said distal handle in a proximal direction.

21. The method for loading prosthesis according to claim 20, the method further including the steps of:
unlocking said locking mechanism and returning said intermediate handle to said distal position to reload said deployed prosthesis on said delivery system and remove or reposition said prosthesis.
